(19) United States
(12) Patent Application Publication
(10) Pub. No.: US 2005/0107838 A1
(43) Pub. Date: May 19, 2005

(54) Subcutaneous cardiac rhythm management with disordered breathing detection and treatment

(76) Inventors: Eric G. Lovett, Mendota Heights, MN (US); Jeffrey E. Stahmann, Ramsey, MN (US); Kent Lee, Fridley, MN (US); Quan Ni, Shoreview, MN (US); Jesse W. Hartley, Lino Lakes, MN (US)

Correspondence Address: CRAWFORD MAUNU PLLC, 1270 NORTHLAND DRIVE, SUITE 390, ST. PAUL, MN, 55120, US

(21) Appl. No.: 10/920,549

(22) Filed: Aug. 17, 2004

Related U.S. Application Data
(63) Continuation-in-part of application No. 10/820,642, filed on Apr. 8, 2004.
(60) Provisional application No. 60/504,229, filed on Sep. 18, 2003.

(51) Int. Cl. A61N 1/365

(52) U.S. Cl. 607/17; 607/42

(57) ABSTRACT

A lead system, coupled to an implantable device, is configured for subcutaneous, non-intrathoracic placement relative to a patient's heart. Cardiac activity detection circuitry is coupled to the lead system and configured to detect cardiac rhythms. Disordered breathing detection circuitry is coupled to the lead system and configured to detect disordered breathing. One or both of cardiac therapy circuitry and disordered breathing therapy circuitry may be coupled to the lead system and configured to delivery therapies to treat disordered breathing. Such therapies include cardiac pacing, diaphragmatic pacing, and hypoglossal nerve stimulation therapies. A patient-external respiratory device, such as a positive airway pressure device, may be configured to deliver a disordered breathing therapy. One or more of a patient-internal drug delivery device, a patient-external drug delivery device, or a gas therapy device may be employed to treat disordered breathing.

### RELATED APPLICATIONS

This application is a continuation-in-part of U.S. patent application Ser. No. 10/820,642 filed Apr. 8, 2004, and claims the benefit of Provisional Patent Application Ser. No. 60/504,229, filed on Sep. 18, 2003, to which priority is claimed pursuant to 35 U.S.C. §119(e), and both of which are hereby incorporated herein by reference.

### FIELD OF THE INVENTION

The present invention relates generally to implantable medical devices and, more particularly, to subcutaneous systems and methods for detecting cardiac and/or disordered breathing activity and treating adverse cardiac and/or disordered breathing conditions.

### BACKGROUND OF THE INVENTION

The healthy heart produces regular, synchronized contractions. Rhythmic contractions of the heart are normally initiated by the sinoatrial (SA) node, which are specialized cells located in the upper right atrium. The SA node is the normal pacemaker of the heart, typically initiating 60-100 heartbeats per minute. When the SA node is pacing the heart normally, the heart is said to be in normal sinus rhythm.

If the heart's electrical activity becomes uncoordinated or irregular, the heart is denoted to be arrhythmic. Cardiac arrhythmia impairs cardiac efficiency and may be a potential life-threatening event. Cardiac arrhythmias have a number of etiological sources, including tissue damage due to myocardial infarction, infection, or degradation of the heart's ability to generate or synchronize the electrical impulses that coordinate contractions.

Bradycardia occurs when the heart rhythm is too slow. This condition may be caused, for example, by impaired function of the SA node, denoted sick sinus syndrome, or by delayed propagation or blockage of the electrical impulse between the atria and ventricles. Bradycardia produces a heart rate that is too slow to maintain adequate circulation.

When the heart rate is too rapid, the condition is denoted tachycardia. Tachycardia may have its origin in either the atria or the ventricles. Tachycardias occurring in the atria of the heart, for example, include atrial fibrillation and atrial flutter. Both conditions are characterized by rapid contractions of the atria. Besides being hemodynamically inefficient, the rapid contractions of the atria may also adversely affect the ventricular rate.

Ventricular tachycardia occurs, for example, when electrical activity arises in the ventricular myocardium at a rate more rapid than the normal sinus rhythm. Ventricular tachycardia may quickly degenerate into ventricular fibrillation. Ventricular fibrillation is a condition denoted by extremely rapid, uncoordinated electrical activity within the ventricular tissue. The rapid and erratic excitation of the ventricular tissue prevents synchronized contractions and impairs the heart's ability to effectively pump blood to the body, which is a fatal condition unless the heart is returned to sinus rhythm within a few minutes.

Implantable cardiac rhythm management systems have been used as an effective treatment for patients with serious arrhythmias. These systems typically include one or more leads and circuitry to sense signals from one or more interior and/or exterior surfaces of the heart. Such systems also include circuitry for generating electrical pulses that are applied to cardiac tissue at one or more interior and/or exterior surfaces of the heart. For example, leads extending into the patient's heart are connected to electrodes that contact the myocardium for sensing the heart's electrical signals and for delivering pulses to the heart in accordance with various therapies for treating the arrhythmias described above.

Implantable cardioverter/defibrillators (ICDs) have been used as an effective treatment for patients with serious cardiac arrhythmias. For example, a typical ICD includes one or more endocardial leads to which at least one defibrillation electrode is connected. Such ICDs are capable of delivering high-energy shocks to the heart, interrupting the ventricular tachyarrythmia or ventricular fibrillation, and allowing the heart to resume normal sinus rhythm. ICDs may also include pacing functionality.

People with severe cardiopulmonary deficiencies, such as those associated with chronic heart failure and other cardiopulmonary maladies, are particularly susceptible to morbidities associated with disordered breathing conditions such as sleep apnea. Disordered breathing may be caused by a wide spectrum of respiratory conditions involving the disruption of the normal respiratory cycle. Although disordered breathing often occurs during sleep, the condition may also occur while the patient is awake. Respiratory disruption can be particularly serious for patients concurrently suffering from cardiovascular deficiencies, such as congestive heart failure. Unfortunately, disordered breathing is often undiagnosed. If left untreated, the effects of disordered breathing may result in serious health consequences for the patient.

Various types of disordered respiration have been identified, including, for example, apnea, hypopnea, dyspnea, hyperpnea, tachypnea, and periodic breathing, including Cheyne-Stokes respiration (CSR). Apnea is a fairly common disorder characterized by periods of interrupted breathing. Apnea is typically classified based on its etiology. One type of apnea, denoted obstructive apnea, occurs when the patient's airway is obstructed by the collapse of soft tissue in the rear of the throat. Central apnea is caused by a derangement of the central nervous system control of respiration. The patient ceases to breathe when control signals from the brain to the respiratory muscles are absent or interrupted. Mixed apnea is a combination of the central and obstructive apnea types. Regardless of the type of apnea, people experiencing an apnea event stop breathing for a period of time. The cessation of breathing may occur repeatedly during sleep, sometimes hundreds of times a night and sometimes for a minute or longer.

### SUMMARY OF THE INVENTION

The present invention is directed to systems and methods for detecting cardiac activity and disordered breathing from subcutaneous, non-intrathoracic locations relative to a heart of a patient. Systems and methods of the present invention are further directed to delivery of therapies for treating abnormal cardiac conditions and detected disordered breathing.

According to one embodiment, a system includes a lead system configured for subcutaneous, non-intrathoracic placement relative to a patient's heart. The system further includes an implantable device comprising cardiac activity detection circuitry and disordered breathing detection circuitry. The cardiac activity detection circuitry is coupled to the lead system and configured to detect cardiac rhythms, and the disordered breathing detection circuitry is coupled to the lead system and configured to detect disordered breathing.

The implantable device may further include one or both of cardiac therapy circuitry and disordered breathing therapy circuitry respectively coupled to the lead system. In one embodiment, the cardiac therapy circuitry is configured to deliver a cardiac therapy to treat detected disordered breathing. In another embodiment, the disordered breathing therapy circuitry includes circuitry to coordinate delivery of a diaphragmatic pacing therapy. In a further embodiment, the disordered breathing therapy circuitry is coupled to a hypoglossal nerve lead and includes circuitry to coordinate delivery of a hypoglossal nerve stimulation therapy.

The system may further include a patient-external respiratory device, such as a positive airway pressure device, configured to deliver a disordered breathing therapy to the patient. The system may also include one or more of a patient-internal drug delivery device, a patient-external drug delivery device, or a gas therapy device. The system may include one or both of an accelerometer and transthoracic impedance sensor configured to detect the patient's respiration.

Each of the implantable device and the respiratory device may include communication circuitry configured to facilitate communication between the implantable device and the respiratory device. In another embodiment, the system includes a patient-external processing system communicatively coupled to the implantable device and the respiratory device. The processing system is configured to cooperate with one or both of the implantable device and the respiratory device to coordinate one or more of patient monitoring, diagnosis, and therapy.

In accordance other embodiments, methods involve detecting cardiac activity of a patient from subcutaneous, non-intrathoracic locations, and sensing, from one or more subcutaneous, non-intrathoracic locations, one or more physiologic parameters associated with respiration of the patient. Methods further involve determining presence of disordered breathing using the sensed one or more physiologic parameters.

Methods of the present invention may involve delivering a disordered breathing therapy in response to determining presence of disordered breathing. Such therapies may involve delivering one or more of a cardiac therapy, a diaphragmatic pacing therapy, a hypoglossal nerve stimulation therapy, a drug therapy, or a gas therapy in response to determining presence of disordered breathing.

The above summary of the present invention is not intended to describe each embodiment or every implementation of the present invention. Advantages and attainments, together with a more complete understanding of the invention, will become apparent and appreciated by referring to the following detailed description and claims taken in conjunction with the accompanying drawings.

### BRIEF DESCRIPTION OF THE DRAWINGS

FIG. 1A is a block diagram depicting a subcutaneous system configurable for monitoring, diagnosing, and/or treating cardiac and/or disordered breathing events or conditions in accordance with embodiments of the present invention;

FIG. 1B is a block diagram illustrating various components of a transthoracic cardiac sensing and/or stimulation system that provides for disordered breathing detection and/or treatment in accordance with an embodiment of the present invention;

FIGS. 1C and 1D are views of a transthoracic cardiac sensing and/or stimulation device as implanted in a patient in accordance with an embodiment of the present invention;

FIG. 1E is a block diagram illustrating various components of a transthoracic cardiac sensing and/or stimulation device in accordance with an embodiment of the present invention;

FIGS. 2A-2C are diagrams illustrating various components of a transthoracic cardiac sensing and/or stimulation device located in accordance with embodiments of the invention;

FIGS. 3A-3C are diagrams illustrating electrode subsystem placement relative to a heart in accordance with embodiments of the invention;

FIG. 4 is a flow chart illustrating a brain state algorithm based on signals from an EEG sensor in accordance with embodiments of the invention;

FIG. 5A is a graph of a normal respiration signal measured by a transthoracic impedance sensor that may be utilized for monitoring, diagnosis and/or therapy in accordance with embodiments of the invention;

FIG. 5B is a respiration signal graph illustrating respiration intervals used for disordered breathing detection according to embodiments of the invention;

FIG. 5C is a graph of a respiration signal illustrating various intervals that may be used for detection of apnea in accordance with embodiments of the invention;

FIG. 6 is a respiration graph illustrating abnormally shallow respiration utilized in detection of disordered breathing in accordance with embodiments of the invention;

FIG. 7 is a flow chart illustrating a method of apnea and/or hypopnea detection according to embodiments of the invention;

FIG. 8 illustrates a medical system including an implantable subcutaneous cardiac rhythm management device that cooperates with a patient-external respiration therapy device to provide coordinated patient monitoring, diagnosis and/or therapy in accordance with an embodiment of the invention; and

FIG. 9 is a block diagram of a medical system that may be used to implement coordinated patient monitoring, diagnosis, and/or therapy in accordance with embodiments of the invention.

While the invention is amenable to various modifications and alternative forms, specifics thereof have been shown by way of example in the drawings and will be described in detail below. It is to be understood, however, that the intention is not to limit the invention to the particular embodiments described. On the contrary, the invention is intended to cover all modifications, equivalents, and alternatives falling within the scope of the invention as defined by the appended claims.

### DETAILED DESCRIPTION OF VARIOUS EMBODIMENTS

In the following description of the illustrated embodiments, references are made to the accompanying drawings, which form a part hereof, and in which is shown by way of illustration, various embodiments in which the invention may be practiced. It is to be understood that other embodiments may be utilized, and structural and functional changes may be made without departing from the scope of the present invention.

An implanted device according to the present invention may include one or more of the features, structures, methods, or combinations thereof described hereinbelow. For example, a cardiac monitor, cardiac stimulator or respiratory device may be implemented to include one or more of the advantageous features and/or processes described below. It is intended that such a monitor, stimulator, respiratory device or other implanted, partially implanted, or external device need not include all of the features described herein, but may be implemented to include selected features that provide for useful structures and/or functionality. Such a device or system may be implemented to provide a variety of diagnostic and/or therapeutic functions.

A significant percentage of people between the ages of 30 and 60 experience some symptoms of disordered breathing. Disordered breathing primarily occurs during sleep, and is associated with excessive daytime sleepiness, systemic hypertension, increased risk of stroke, angina, and myocardial infarction. Disordered breathing is particularly prevalent among congestive heart failure patients, and may contribute to the progression of heart failure.

Embodiments of the invention are directed to methods and devices that provide for detection and/or monitoring of cardiac and respiratory activity. Further embodiments of the invention are directed to methods and devices that provide for treatment of adverse cardiac and/or respiratory conditions. In one particular embodiment, for example, an implantable transthoracic cardiac sensing and/or stimulation (ITCS) device is implemented to detect/monitor adverse cardiac and/or respiratory conditions, and may be configured to deliver an appropriate therapy in response thereto.

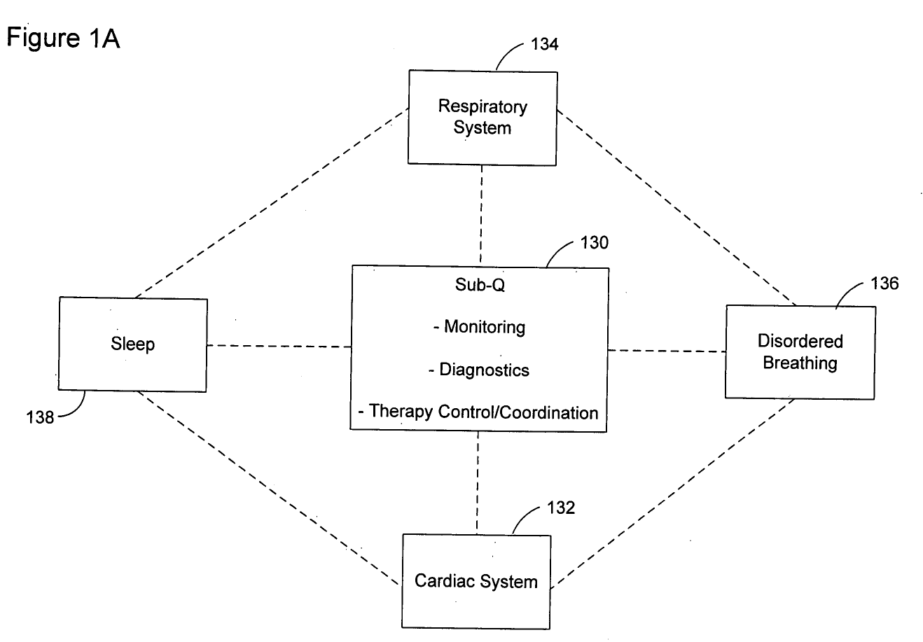

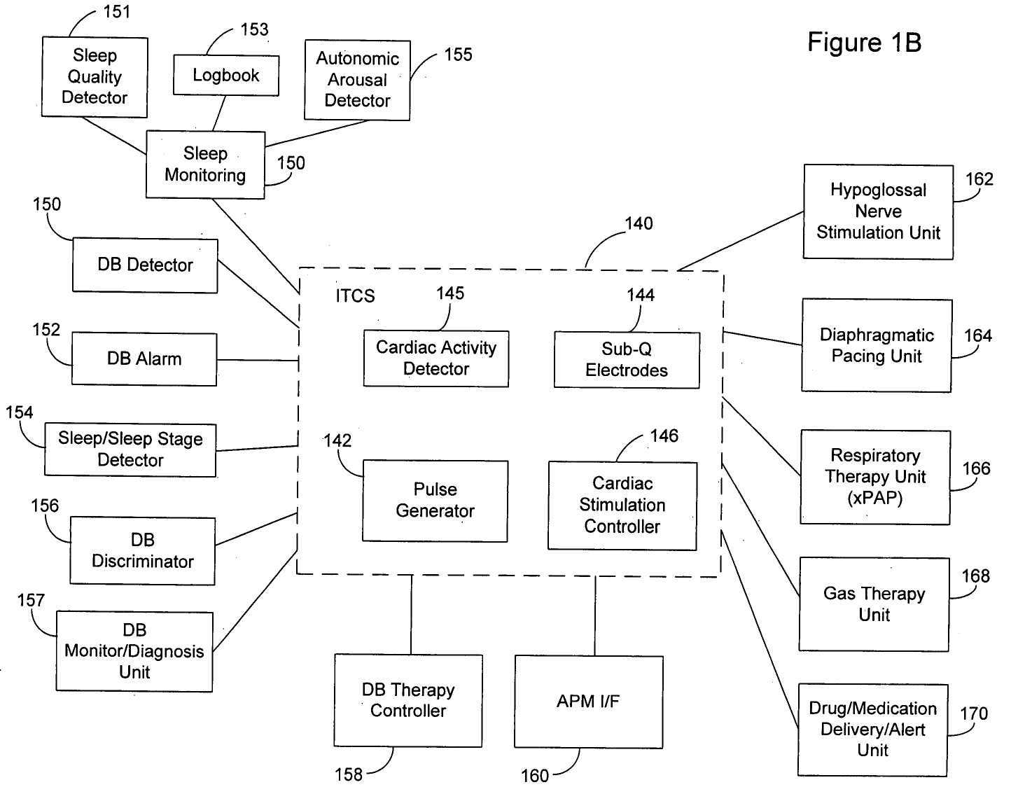

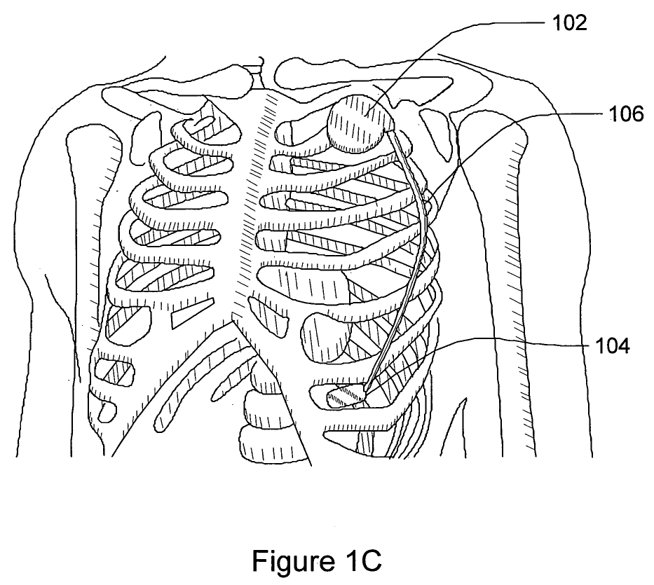

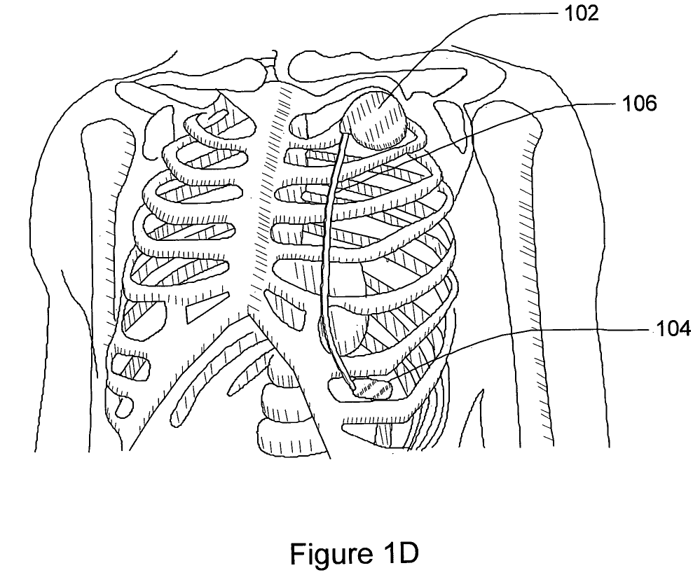

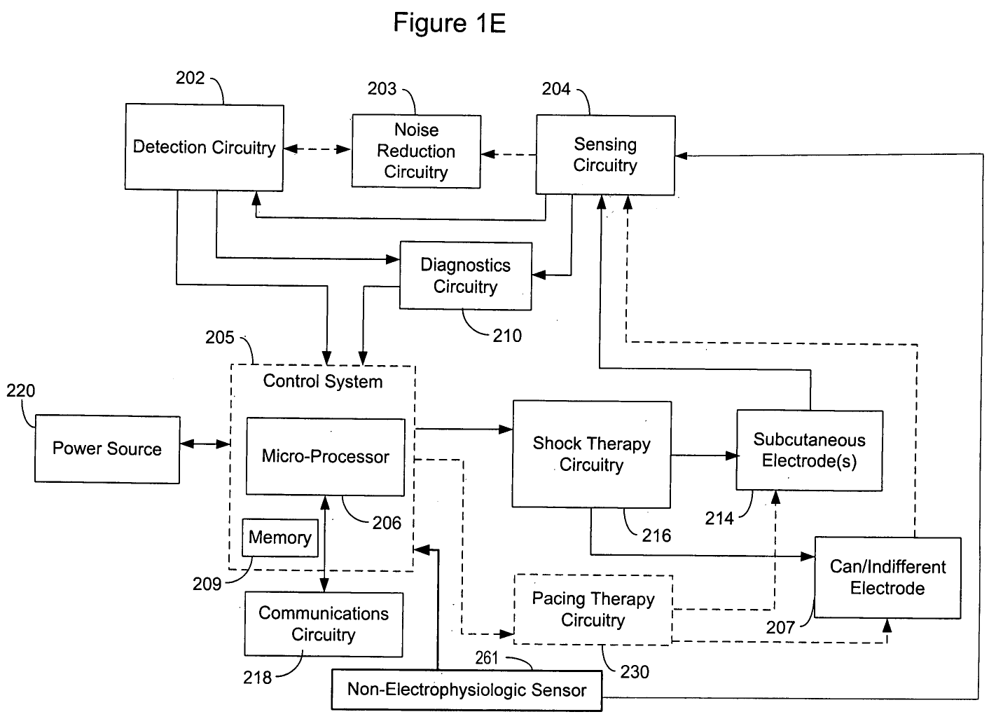

FIG. 1A is a block diagram illustrating a subcutaneous system 130, such as an ITCS, configurable for monitoring, diagnosing, and/or treating cardiac and/or disordered breathing events/conditions in accordance with embodiments of the present invention. The subcutaneous system 130 is implemented to sense activity of both the cardiac system 132 and the respiratory system 134. Using appropriate sensors, the subcutaneous system 130 may be implemented to detect and monitor a variety of disordered breathing conditions 136, including sleep and non-sleep related disordered breathing conditions. The subcutaneous system 130 may further be implemented to detect sleep 138, and may further be implemented to detect stages of patient sleep. A subcutaneous system 130 so implemented may be configured to perform a variety of sensing, monitoring, diagnosing, and therapy control/coordination functions, alone or in cooperation with other devices, such as an external respiratory device, an advanced patient management system, or other systems as described herein and in the references respectively incorporated herein.

FIG. 1B is a block diagram illustrating various components of a transthoracic cardiac sensing and/or stimulation system that provides for disordered breathing detection and/or treatment in accordance with embodiments of the present invention. It is understood that the components/blocks shown in FIG. 1B represent non-limiting examples of various functional or structural elements that may be incorporated as part of an ITCS system of the present invention. It is further understood that embodiments of an ITCS system of the present invention may incorporate one, several, or all of the functional or structural elements depicted in FIG. 1B, and that a wide variety of device/system configurations are contemplated. Also, the individual blocks shown in FIG. 1B are for purposes of clarity, and are not intended to imply that such blocks are independent functional units. It is understood that the functions and/or structures associated with individual blocks may be performed by, or incorporated within, common blocks or a signal block, such as in the ITCS block 140.

In general terms, cardiac activity and disordered breathing (e.g., sleep disordered breathing and wakeful disordered breathing) may be detected, monitored, and/or treated with use of a subcutaneous cardiac monitoring and/or energy delivery device, such as an ITCS device 140, in accordance with the present invention. An ITCS device 140 may be implanted under the skin in the chest region of a patient. The ITCS device 140 may, for example, be implanted subcutaneously such that all or selected elements of the device are positioned on the patient's front, back, side, or other body locations suitable for sensing cardiac activity and delivering cardiac stimulation therapy. It is understood that elements of the ITCS device 140 may be located at several different body locations, such as in the chest, abdominal, or subclavian region with electrode elements respectively positioned at different regions near, around, or on the heart.

The primary housing (e.g., the active or non-active can) of the ITCS device 140, for example, may be configured for positioning outside of the rib cage at an intercostal or subcostal location, within the abdomen, or in the upper chest region (e.g., subclavian location, such as above the third rib). In one implementation, one or more electrodes 144 may be located on the primary housing and/or at other locations about, but not in direct contact with the heart, great vessel or coronary vasculature. A pulse generator 142 and a cardiac stimulation controller 146 are disposed in the primary housing. The cardiac stimulator controller 146 determines and coordinates appropriate cardiac and/or respiratory therapy to be delivered to a patient, and the pulse generator 142 produces the appropriate energy waveforms associated with a selected therapy. Also disposed in the primary housing is a cardiac activity detector 145 configured to detect normal and abnormal (e.g., arrhythmia) cardiac activity.

In a further implementation, one or more subcutaneous electrode subsystems or electrode arrays 144 may be used to sense cardiac activity and deliver cardiac stimulation energy in an ITCS device configuration employing an active can or a configuration employing a non-active can. Electrodes 144 may be situated at anterior and/or posterior locations relative to the heart. Examples of useful electrode locations and features that may be incorporated in various embodiments of the present invention are described in commonly owned, co-pending U.S. patent application Ser. No. 10/465,520 filed Jun. 19, 2003, entitled “Methods and Systems Involving Subcutaneous Electrode Positioning Relative to a Heart”; Ser. No. 10/795,126 filed Mar. 5, 2004, entitled “Wireless ECG In Implantable Devices”; and Ser. No. 10/738,608 filed Dec. 17, 2003, entitled “Noise Canceling Cardiac Electrodes,” which are hereby incorporated herein by reference.

The ITCS device 140 depicted in FIG. 1B may be configured in a manner described herein or may have other configurations. An ITCS device 140 of the present invention may be implemented to include one or more of cardiac and/or respiratory detection/monitoring circuitry (e.g., for cardiac activity, breathing patterns such as from transthoracic impedance signals, heart sounds, blood gas/chemistry such as oxygen saturation and/or pH), cardiac and respiratory diagnostics circuitry, and cardiac and respiratory therapy circuitry. An ITCS device 140 of the present invention may be implemented to provide for upgradeability in terms of functionality and/or configuration. For example, an ITCS device 140 may be implemented as an upgradeable or reconfigurable cardiac/respiratory monitor or stimulation device, such as in a manner described in one or more of commonly owned, co-pending U.S. patent application Ser. Nos. 10/462,001 (Attorney Docket No. GUID.612PA) filed Jun. 13, 2003; Ser. No. 10/821,248 (Attorney Docket No. GUID.618PA) filed Jun. 8, 2004; and Ser. No. 10/785,431 (Attorney Docket No. GUID.048US01) filed Feb. 24, 2004. An ITCS device 140 may be implemented to provide a variety of cardiac therapies, such as is described in previously incorporated U.S. patent application Ser. No. 10/820, 642. Additional embodiments and features of an ITCS device of the present invention are described in greater detail hereinbelow.

An ITCS device 140 in accordance with embodiments of the present invention provides for patient breathing monitoring and disordered breathing detection and/or prediction. Such embodiments may further provide treatment for detected or predicted disordered breathing events or conditions, as determined by a therapy controller 158 or in response to an externally generated command signal (such as received from an advanced patient management system via APM interface 160). Detection and treatment of disordered breathing and/or respiratory conditions may be facilitated by use of an ITCS device 140 having appropriate sensing/detection/therapy delivery capabilities, or by cooperative use of an ITCS device 140 and an external respiration detection and/or therapy delivery device or via an advanced patient management system via APM interface 160.

Various therapies have been used to treat disordered breathing, including both central and obstructive types. Obstructive sleep apnea has been associated with prolapse of the tongue and its surrounding structure into the pharynx, thus occluding the respiratory pathway. A commonly prescribed treatment for obstructive apnea is continuous positive airway pressure (CPAP). A CPAP device delivers air pressure through a nasal mask worn by the patient. The application of continuous positive airway pressure keeps the patient's throat open, reducing or eliminating the obstruction causing the apnea.

Positive airway pressure devices may be used to deliver a variety of respiration therapies, including, for example, continuous positive airway pressure (CPAP), bi-level positive airway pressure (bi-level PAP), proportional positive airway pressure (PPAP), auto-titrating positive airway pressure, ventilation, gas or oxygen therapies. All types of positive airway pressure devices are referred to generically herein as xPAP devices. Some positive airway pressure devices may also be configured to provide both positive and negative pressure, such that negative pressure is selectively used (and de-activated) when necessary, such as when treating Cheyne-Stokes breathing, for example. The term CPAP will be used herein as a generic term for any device using forms of positive airway pressure (and negative pressure when necessary), whether continuous or otherwise.

In various implementations, detection of sleep disordered breathing may be used to initiate an externally delivered respiration therapy, such as by using a CPAP device 166. A CPAP device 166 delivers air pressure through a nasal mask worn by the patient. The application of continuous positive airway pressure keeps the patient's throat open, reducing or eliminating the obstruction causing the apnea. In one embodiment of the invention, detection of sleep disordered breathing may initiate or modify CPAP therapy delivered to the patient.

In further implementations, both cardiac therapy and positive airflow pressure therapy may be delivered to the patient, via ITCS and CPAP devices 140, 166, respectively. Methods and systems for providing coordinated therapies involving cardiac electrical stimulation therapy and external respiration therapy for the treatment of disordered breathing are described in commonly owned U.S. Patent Application Ser. No. 60/504,561, filed Aug. 18, 2003 entitled “Treatment of Disordered Breathing Using a Combination of Respiratory and Cardiac Therapies,” which is hereby incorporated herein by reference. A variety of embodiments for delivering CPAP therapy, which may operate in cooperation with an ITCS device 140, are disclosed in commonly owned, co-pending U.S. Patent Application No. 60/504,229, filed on Sep. 18, 2003 under Attorney Docket No. GUID.151P1, which is hereby incorporated herein by reference.

An ITCS device 140 according to embodiments of the present invention may be configured to determine and/or monitor the sleep state of a patient, which may be useful for assessing disordered breathing during patient sleep. A patient's sleep state may be determined by analyzing one or more patient conditions indicative of sleep, such as by use of a sleep monitor 150. The sleep monitor 150 may be part of the ITCS device 140 or may be an external system that communicates with the ITCS device 140. The sleep monitor 150 may detect sleep on the basis of changes in the patient's heart rate, activity, respiration, or a combination of these conditions and/or other conditions. The conditions used to detect sleep may be sensed using a combination of implantable or patient-external sensors and devices, such as impedance sensors, EEG sensors, EMG sensors, snoring sensors, acoustic transducers, motion sensors, and other sensors useful for detecting sleep and/or sleep staging. Examples of sleep state detection and classification systems and methods are disclosed in commonly owned co-pending U.S. patent application Ser. No. 10/643,006 filed on Aug. 18, 2003, which is hereby incorporated herein by reference.

In one embodiment, and as described in previously incorporated U.S. Ser. No. 10/643,006, an ITCS device 140 incorporates or is otherwise coupled to a sensor system 150 configured to sense sleep-related signals. The sensor system 150 includes at least one sensor configured to sense a sleep-wake condition of a patient and at least one sensor configured to sense a condition associated with REM sleep. A classification system is coupled to the sensor system 150 and configured to classify sleep states based on the sensed sleep-related signals. One or both of the sensor system 150 and the classification system is implantable or includes an implantable component, such as a component (e.g., processor) of the ITCS device 140. The sensor configured for sensing the patient's sleep-wake condition may include an accelerometer or a transthoracic impedance sensor. The sensor configured for sensing a condition associated with REM sleep may include a skeletal muscle tone sensor, such as an electromyogram (EMG) sensor, a brain wave sensor such as an electroencephalogram (EEG) sensor, a mechanical strain gauge, or a mechanical force sensor, for example.

Other embodiments of the present invention may provide for organizing information related to sleep and/or events occurring during sleep, such as by use of a logbook 153. One embodiment of the invention involves an automated method for collecting and organizing information associated with sleep. This approach involves detecting sleep and acquiring information associated with sleep. The acquired information is organized as a sleep logbook 153. At least one of detecting sleep, acquiring the information associated with sleep, and organizing the acquired information is preferably performed at least in part implantably, which may be performed by the ITCS device 140.

Another embodiment involves organizing sleep-related information using the sleep logbook 153. Information associated with one or more sleep periods is acquired, such as by use of the ITCS device 140. The information associated with the one or more sleep periods is organized in the sleep logbook 153. For example, a data acquisition unit may be configured to acquire sleep information related to sleep. A processor (e.g., of the ITCS device 140) is coupled to the a sleep detector 154 and the data acquisition unit. The processor organizes the acquired sleep information as a sleep logbook entry in the logbook 153. A user interface is provided for accessing the sleep logbook 153. Additional details of an ITCS device embodiment that includes sleep logbook functionality are disclosed in commonly owned, co-pending U.S. patent application entitled “Sleep Logbook,” filed concurrently herewith under Attorney Docket GUID.182PA, which is hereby incorporated herein by reference.

The sleep monitor 150 shown in FIG. 1B may incorporate or otherwise be coupled to a sleep quality detector 151. The sleep quality detector 151 includes a detector system configured to detect physiological and non-physiological conditions associated with sleep quality and a data collection system for collecting sleep quality data based on the detected conditions. The data collection system may be part of the ITCS device 140 or other patient-internal or external system.

The sleep quality detector 151 is configured to evaluate sleep quality. For example, a processor of the sleep quality detector 151 may be configured to determine metrics based on the detected conditions. The metrics may include one or more metrics associated with sleep, one or more metrics associated with events that disrupt sleep, and at least one composite sleep quality metric based on the one or more metrics associated with sleep and the one or more metrics associated with events that disrupt sleep. The processor may further determine a composite sleep quality metric as a function of the metrics associated with sleep and the metrics associated with events that disrupt sleep.

In another embodiment, the sleep quality detector 151 detects one or more patient conditions associated with sleep quality during a period of wakefulness and collects sleep quality data based on the detected conditions. The sleep quality detector 151 evaluates the sleep quality of the patient using the collected sleep quality data. Additional details of an ITCS device embodiment that includes sleep quality detection functionality are disclosed in commonly owned, co-pending U.S. patent application entitled “Sleep Quality Data Collection and Evaluation,” filed Aug. 18, 2003 and receiving Ser. No. 10/642,998 (GUID.058PA), which is hereby incorporated herein by reference.

In another example, the patient's sleep quality may be evaluated by determining the patient's activity level while the patient is awake. The activity level of the patient during the day may provide important information regarding the patient's sleep quality. For example, if the patient is very inactive during periods of wakefulness, this may indicate that the patient's sleep is of inadequate quality or duration. Such information may also be used in connection with assessing the efficacy of a particular sleep disorder therapy and/or adjusting the patient's sleep disorder therapy. Methods and systems for determining the patient's activity level and generally assessing the well-being of a patient are described in commonly owned U.S. Pat. No. 6,021,351 which is incorporated herein by reference.

As is further shown in FIG. 1B, an ITCS device 140 may incorporate or otherwise by coupled to an autonomic arousal detector 155. The autonomic arousal detector 155 acquires sleep information including autonomic arousal events. The autonomic arousal detector 155 senses one or more physiological conditions modulated by a patient's autonomic arousal response. Autonomic arousal events occurring during sleep are detected based on the one or more sensed signals. For example, an arousal signal modulated by changes in muscle tone associated with autonomic arousal is sensed using an implantable sensor of the autonomic arousal detector 155. Autonomic arousal events are detected based on the arousal signal.

According to other embodiments, one or both of a signal modulated by brainwave activity associated with an autonomic arousal response and a signal modulated by changes in muscle tone associated with the autonomic arousal response are sensed by the autonomic arousal detector 155. Autonomic arousal events are detected by the autonomic arousal detector 155 based on at least one of the brainwave signal and the muscle tone signal. Additional details of an ITCS device embodiment that includes autonomic arousal detection functionality are disclosed in commonly owned, co-pending U.S. patent application entitled “Autonomic Arousal Detection System and Method,” filed concurrently herewith under Attorney Docket GUID.106PA, which is hereby incorporated herein by reference.

In accordance with various embodiments, after determining that the patient is asleep, the ITCS device 140 monitors one or more respiration-related signals to detect sleep disordered breathing. A disordered breathing detector 150 may detect disordered breathing by sensing and analyzing various physiological and/or non-physiological conditions associated with disordered breathing. Detection of disordered breathing may involve comparing one condition or multiple conditions to one or more thresholds or other indices indicative of disordered breathing.

In one embodiment, the DB detector 150 detects disordered breathing by analyzing the patient's respiration patterns as described in more detail below. Patient respiration may be sensed using an implanted or patient-external sensor. For example, implantable methods of sensing patient respiration may involve the use of an implantable transthoracic impedance sensor and/or an implantable blood gas sensor. Patient-external methods of sensing patient respiration may involve the use of devices such as a respiratory belt or external air-flow meter. Communications between an internal ITCS device 140 and one or more patient-external sensors or systems may be facilitated using a variety of known approaches, such as various wireless communications protocols (e.g., short-range RF protocols, such as a Bluetooth protocol).

If disordered breathing is detected during sleep, the DB therapy controller 158 of the ITCS device 140 may perform a number of operations. Such operations may vary depending on the particular features provided or otherwise enabled by a given ITCS device 140 for a particular patient. These operations may involve relatively simple processes (e.g., storing and/or telemetering disordered breathing sensor data), moderately complex processes (e.g., classifying and/or confirming disordered breathing, reporting or alerting disordered breathing events locally or remotely via an advanced patient management system (APM), or more sophisticated processes (treatment of disordered breathing by ITCS device 140 or a combination of the ITCS device 140 and another implantable or patient-external device, such as a CPAP device 166).

By way of example, an alarm unit 152 of the ITCS device 140 may generate an alert to arouse the patient or patient's caregiver, such as an auditory tone, a vibration, and/or other appropriate indicators. The alert may be generated immediately or otherwise contemporaneously with detection of the sleep disordered breathing.

In one scenario, the alert is directed to the patient, for example, to awaken the patient from sleep and thus end the sleep apnea episode. In another scenario, the alert is directed to the patient's caregiver, so that the caregiver can wake the patient or provide an appropriate therapy, for example. In one implementation, a signal may be transmitted from an implantable device to a patient monitoring station used by the patient's caregiver. The patient monitoring station may generate an alert, e.g., an audible alarm or visual alarm, responsive to the detection of the sleep disordered breathing. Additional details of an ITCS device embodiment that includes sleep disordered breathing alarm functionality are disclosed in commonly owned, co-pending U.S. patent application entitled “Sleep Disordered Breathing Alert System,” filed Mar. 4, 2004 under Attorney Docket No. GUID.100PA and assigned Ser. No. 10/793,177, which is hereby incorporated herein by reference.

In another scenario, upon detection of sleep disordered breathing, the DB therapy controller 158 of the ITCS device 140 may initiate delivery of an appropriate therapy to alleviate the disordered breathing. Various types of therapies may be delivered by the ITCS device 140. In one implementation, detection of sleep disordered breathing may trigger the application of cardiac electrical stimulation therapy for disordered breathing, such as may be coordinated by the cardiac stimulation controller 146 of the ITCS device 140. Methods and systems for providing cardiac electrical stimulation therapy for sleep disordered breathing are described in commonly owned U.S. patent application Ser. No. 10/643,203, filed Aug. 18, 2003 and hereby incorporated herein by reference.

Cardiac pacing during periods of sleep or wakefulness may reduce incidents of disordered breathing. Various embodiments discussed herein relate to systems and methods for delivering and adapting an effective cardiac electrical therapy to mitigate disordered breathing. Such a therapy may be adapted, for example, to achieve an overall level of therapy efficacy. The therapy may be adapted to provide a tiered therapy capable of achieving a variety of therapeutic goals. For example, the therapy may be adapted to prevent further disordered breathing episodes, to terminate a detected disordered breathing episode, and/or to achieve a desired reduction in the overall frequency and/or severity of disordered breathing episodes. The cardiac electrical therapy may also be adapted to provide a therapy that balances therapeutic goals with conservation of device life, for example.

The therapy may be adapted to adjust the impact of the therapy on the patient, for example, to reduce the impact of the therapy on the patient. In adapting a reduced impact therapy, the system may take into account various conditions for evaluating the impact of the therapy on the patient. For example, conditions such as patient comfort, as indicated by patient feedback, undesirable side effects, stress on physiological systems involved in the disordered breathing therapy, interaction with cardiac pacing algorithms, e.g., bradycardia pacing, cardiac resynchronization pacing and/or anti-tachycardia pacing, as determined by interactive effects of the disordered breathing therapy with cardiac pacing, and/or sleep quality, as measured by one or more sleep quality indices, may be taken into account to adapt a therapy that reduces an impact of the therapy on the patient.

In addition, impact to the patient may involve a decreased useful service life of an implantable therapeutic device used to deliver disordered breathing therapy and/or pacing therapy for cardiac dysfunction. For example, a level of disordered breathing therapy may be unacceptably high if the energy requirements of the therapy result in an excessively decreased device service life. In this situation, early device removal and replacement produces a negative impact to the patient. Cardiac electrical therapy to mitigate disordered breathing may be adapted based on a projected decrease in device lifetime.

The following commonly owned U.S. patents applications, some of which have been identified above, are hereby incorporated by reference in their respective entireties: U.S. patent application Ser. No. 10/309,770 (Docket Number GUID.064PA), filed Dec. 4, 2002, U.S. patent application Ser. No. 10/309,771 (Docket Number GUID.054PA), filed Dec. 4, 2002, U.S. patent application entitled “Prediction of Disordered Breathing,” identified by Docket Number GUID.088PA and concurrently filed with this patent application, U.S. patent application entitled “Adaptive Therapy for Disordered Breathing,” identified by Docket Number GUID.059PA and filed concurrently with this patent application, U.S. patent application entitled “Sleep State Classification,” identified by Docket Number GUID.060PA and filed concurrently with this patent application, and U.S. patent application entitled “Therapy Triggered by Prediction of Disordered Breathing,” identified by Docket Number GUID.103PA and filed concurrently with this patent application. An ITCS device of the present invention may be implemented to include selected features, functions, and structures described in these and other applications and patents incorporated herein by reference.

In another embodiment of the invention, an ITCS device 140 may coordinate or participate in the classification of the origin of disordered breathing events and/or discrimination between disordered breathing origin types. In one approach, a disordered breathing discriminator 156 is configured to classify disordered breathing in a patient. The DB detector 150 detects a disordered breathing event and further senses motion associated with respiratory effort during the disordered breathing event. The disordered breathing event is classified by the DB discriminator 156 based on the sensed motion. For example, the DB discriminator 156 discriminates between central and obstructive disordered breathing based on sensed motion associated with respiratory effort during the disordered breathing event. Additional details of an ITCS device embodiment that includes disordered breathing discrimination functionality are disclosed in commonly owned, co-pending U.S. patent application Ser. No. 10/824,776, filed Apr. 15, 2004 under Attorney Docket GUID.124PA, and entitled “System and Method for Discrimination Of Central And Obstructive Disordered Breathing Events,” which is hereby incorporated herein by reference.

According to a further embodiment, an ITCS device 140 may include a disordered breathing monitor and/or diagnosis unit 157. In one configuration, the DB monitor and/or diagnosis unit 157 of the ITCS device 140 includes or is otherwise coupled to a respiratory event logbook system, which includes an event detector configured to detect or predict a respiratory event affecting the patient. A data acquisition unit is coupled to the event detector and is configured to collect medical information associated with the respiratory event responsive to the detection or prediction of the respiratory event. A processor of the DB monitor and/or diagnosis unit 157 is configured to organize the collected medical information associated with the respiratory event as a respiratory event log entry.

In one embodiment, a respiratory event of a patient is detected or predicted. Responsive to the detection or prediction of the respiratory event, collection of medical information associated with the respiratory event is initiated by the DB monitor and/or diagnosis unit 157. The medical information is collected and organized as a respiratory event log entry. A user interface is provided for accessing the respiratory logbook.

In another embodiment, a respiratory event is predicted. The DB monitor and/or diagnosis unit 157 collects information associated with conditions affecting the patient prior to the occurrence of the respiratory event. The respiratory event is detected, and the DB monitor and/or diagnosis unit 157 collects information during the respiratory event. The collected information is organized as a respiratory event log entry.

In accordance with another embodiment of the invention, a respiratory event logbook system implemented using the DB monitor and/or diagnosis unit 157 includes an event detector configured to detect or predict a respiratory event. A data acquisition unit is coupled to the event detector and is configured to collect, responsive to the detection or prediction of the respiratory event, respiratory information associated with the event. The DB monitor and/or diagnosis unit 157 also includes, or is coupled to, a processor configured to organize the acquired respiratory information as a respiratory event log entry. Additional details of an ITCS device embodiment that includes respiratory event logbook functionality are disclosed in commonly owned, co-pending U.S. patent application entitled “Medical Event Logbook System and Method,” filed concurrently herewith under Attorney Docket GUID.109PA, which is hereby incorporated herein by reference.

According to one embodiment, snoring sounds generated by a patient may be detected, and the presence of sleep disordered breathing may be determined using the detected snoring sounds. In another embodiment, snoring may be detected from disturbances in a respiration or airflow signal. Snoring sounds or snoring-related respiration/airflow disturbances may be detected internally of the patient, such as by a snore sensor (not shown) in or coupled to the ITCS device 140, or externally of the patient. Determining presence of sleep disordered breathing may be performed internally, by the ITCS device 140, or externally of the patient. Determining presence of sleep disordered breathing may include computing a snoring index developed from the detected snoring. Sleep apnea may be detected using the snoring index. Sleep apnea may be verified using internal or external sensors. In one approach, sleep disordered breathing is detected, such as by use of a minute ventilation sensor, and presence of the sleep disordered breathing may be confirmed using the detected snoring. Additional details of an ITCS device embodiment that detects sleep disordered breathing using snoring sounds are disclosed in previously incorporated U.S. Patent Application No. 60/504,229.

According to other embodiments, detection of sleep disordered breathing by the ITCS device 140 may trigger a muscle stimulation therapy. Prolapse of the tongue muscles has been attributed to diminishing neuromuscular activity of the upper airway. A treatment for obstructive sleep apnea involves compensating for the decreased muscle activity by electrical activation of the tongue muscles. The hypoglossal (HG) nerve innervates the protrusor and retractor tongue muscles. An appropriately applied electrical stimulation to the hypoglossal nerve, for example, may prevent backward movement of the tongue, thus preventing the tongue from obstructing the airway. An ITCS 140 device may include or otherwise cooperate with an HG stimulation device 162, and include structures or methods described in U.S. Pat. Nos. 5,540,732 and 5,540,733, both of which are hereby incorporated herein by reference.

By way of example, a stimulation lead may extend from the ITCS device 140 to a nerve that activates at least one of the patient's upper airway muscles. The ITCS device 140 may monitor the patient's respiration, such as by use of a transthoracic impedance sensor. In response to detection of a disordered breathing event, such as sleep apnea, the ITCS device 140 may delivery electrical stimulation to the nerve to terminate the disordered breathing condition, such as by restoring patency in the patient's airway. The electrical stimulation may be delivered synchronously with the onset of inspiration. In another configuration, the HG stimulation device 162 may be a device separate from the ITCS device 140 but communicatively linked thereto via an RF or other communications link.

Another electrical stimulation therapy for treating disordered breathing using an ITCS device 140 involves phrenic nerve pacing, which is also denoted diaphragmatic pacing. This therapy may be delivered by a diaphragmatic pacing unit 164, typically incorporated as part of the pulse generator 142 of the ITCS device 140. The phrenic nerve is generally known as the motor nerve of the diaphragm. It runs through the thorax, along the heart, and then to the diaphragm. Diaphragmatic pacing via an ITCS device 140 involves the use of electrical stimulation of the phrenic nerve to control the patient's diaphragm. The electric stimulus of the phrenic nerve causes the diaphragm to induce a respiratory cycle. Methods and systems of diaphragmatic pacing that may be implemented by an ITCS device 140 of the present invention are described in commonly owned U.S. Pat. No. 6,415,183, which is incorporated herein by reference.

An ITCS device 140 may be implemented to coordinate or otherwise provide physiologic data for, delivery, termination, or adjustment of a gas therapy to the patient. A gas therapy unit 168 typically external to the patient may be controlled by the ITCS device 140, cooperatively by use of the ITCS device 140, or by use of physiologic or other data acquired or processed by the ITCS device 140. In another configuration, the ITCS device 140 may incorporate, control, or otherwise provide data to a drug/medication delivery and/or alert unit 170. The drug/medication delivery unit 170 may be patient-internal or patient-external, and the alert unit 170 is typically patient-external. Various drugs and pharmacological agents may be administered to the patient, such as via an XPAP device 166, nebulizer, IV, or internal drug pump/delivery mechanism. Additional details of ITCS device embodiments that provide and control gas therapy and/or drug delivery/alerting are disclosed in previously incorporated U.S. Patent Application No. 60/504,229.

Referring now to FIGS. 1C and 1D of the drawings, there is shown a configuration of an ITCS device having components implanted in the chest region of a patient at different locations. In the particular configuration shown in FIGS. 1C and 1D, the ITCS device includes a housing 102 within which various cardiac and respiratory sensing, detection, processing, and energy delivery circuitry may be housed. It is understood that the components and functionality depicted in the figures and described herein may be implemented in hardware, software, or a combination of hardware and software. It is further understood that the components and functionality depicted as separate or discrete blocks/elements in the figures may be implemented in combination with other components and functionality, and that the depiction of such components and functionality in individual or integral form is for purposes of clarity of explanation, and not of limitation.

Communications circuitry is disposed within the housing 102 for facilitating communication between the ITCS device and an external communication device, such as a portable or bed-side communication station, patient-carried/worn communication station, or external programmer, for example. The communications circuitry may also facilitate unidirectional or bidirectional communication with one or more external, cutaneous, or subcutaneous physiologic or non-physiologic sensors. The housing 102 is typically configured to include one or more electrodes (e.g., can electrode and/or indifferent electrode). Although the housing 102 is typically configured as an active can, it is appreciated that a non-active can configuration may be implemented, in which case at least two electrodes spaced apart from the housing 102 are employed.

In the configuration shown in FIGS. 1C and 1D, a subcutaneous electrode 104 may be positioned under the skin in the chest region and situated distal from the housing 102. The subcutaneous and, if applicable, housing electrode(s) may be positioned about the heart at various locations and orientations, such as at various anterior and/or posterior locations relative to the heart. The subcutaneous electrode 104 is coupled to circuitry within the housing 102 via a lead assembly 106. One or more conductors (e.g., coils or cables) are provided within the lead assembly 106 and electrically couple the subcutaneous electrode 104 with circuitry in the housing 102. One or more sense, sense/pace or defibrillation electrodes may be situated on the elongated structure of the electrode support, the housing 102, and/or the distal electrode assembly (shown as subcutaneous electrode 104 in the configuration shown in FIGS. 1C and 1D).

In one configuration, the lead assembly 106 is generally flexible and has a construction similar to conventional implantable, medical electrical leads (e.g., defibrillation leads or combined defibrillation/pacing leads). In another configuration, the lead assembly 106 is constructed to be somewhat flexible, yet has an elastic, spring, or mechanical memory that retains a desired configuration after being shaped or manipulated by a clinician. For example, the lead assembly 106 may incorporate a gooseneck or braid system that may be distorted under manual force to take on a desired shape. In this manner, the lead assembly 106 may be shape-fit to accommodate the unique anatomical configuration of a given patient, and generally retains a customized shape after implantation. Shaping of the lead assembly 106 according to this configuration may occur prior to, and during, ITCS device implantation.

In accordance with a further configuration, the lead assembly 106 includes an electrode support assembly, such as an elongated structure that positionally stabilizes the subcutaneous electrode 104 with respect to the housing 102. In this configuration, the rigidity of the elongated structure maintains a desired spacing between the subcutaneous electrode 104 and the housing 102, and a desired orientation of the subcutaneous electrode 104/housing 102 relative to the patient's heart. The elongated structure may be formed from a structural plastic, composite or metallic material, and includes, or is covered by, a biocompatible material. Appropriate electrical isolation between the housing 102 and subcutaneous electrode 104 is provided in cases where the elongated structure is formed from an electrically conductive material, such as metal.

In one configuration, the electrode support assembly and the housing 102 define a unitary structure (e.g., a single housing/unit). The electronic components and electrode conductors/connectors are disposed within or on the unitary ITCS device housing/electrode support assembly. At least two electrodes are supported on the unitary structure near opposing ends of the housing/electrode support assembly. The unitary structure may have an arcuate or angled shape, for example.

According to another configuration, the electrode support assembly defines a physically separable unit relative to the housing 102. The electrode support assembly includes mechanical and electrical couplings that facilitate mating engagement with corresponding mechanical and electrical couplings of the housing 102. For example, a header block arrangement may be configured to include both electrical and mechanical couplings that provide for mechanical and electrical connections between the electrode support assembly and housing 102. The header block arrangement may be provided on the housing 102 or the electrode support assembly. Alternatively, a mechanical/electrical coupler may be used to establish mechanical and electrical connections between the electrode support assembly and housing 102. In such a configuration, a variety of different electrode support assemblies of varying shapes, sizes, and electrode configurations may be made available for physically and electrically connecting to a standard ITCS device housing 102.

It is noted that the electrodes and the lead assembly 106 may be configured to assume a variety of shapes. For example, the lead assembly 106 may have a wedge, chevron, flattened oval, or a ribbon shape, and the subcutaneous electrode 104 may include a number of spaced electrodes, such as an array or band of electrodes. Moreover, two or more subcutaneous electrodes 104 may be mounted to multiple electrode support assemblies 106 to achieve a desired spaced relationship amongst subcutaneous electrodes 104.

An ITCS device may incorporate circuitry, structures and functionality of the subcutaneous implantable medical devices disclosed in commonly owned U.S. Pat. Nos. 5,203,348; 5,230,337; 5,360,442; 5,366,496; 5,397,342; 5,391,200; 5,545,202; 5,603,732; and 5,916,243, which are hereby incorporated herein by reference.

FIG. 1E is a block diagram depicting various components of an ITCS device in accordance with one configuration. According to this configuration, the ITCS device incorporates a processor-based control system 205 which includes a micro-processor 206 coupled to appropriate memory (volatile and non-volatile) 209, it being understood that any logic-based control architecture may be used. The control system 205 is coupled to circuitry and components to sense, detect, and analyze electrical signals produced by the heart and deliver electrical stimulation energy to the heart under predetermined conditions to treat cardiac arrhythmias. In certain configurations, the control system 205 and associated components also provide pacing therapy to the heart. The electrical energy delivered by the ITCS device may be in the form of low energy pacing pulses or high-energy pulses for cardioversion or defibrillation.

Cardiac signals are sensed using the subcutaneous electrode(s) 214 and the can or indifferent electrode 207 provided on the ITCS device housing. Cardiac signals may also be sensed using only the subcutaneous electrodes 214, such as in a non-active can configuration. As such, unipolar, bipolar, or combined unipolar/bipolar electrode configurations as well as multi-element electrodes and combinations of noise canceling and standard electrodes may be employed. The sensed cardiac signals are received by sensing circuitry 204, which includes sense amplification circuitry and may also include filtering circuitry and an analog-to-digital (A/D) converter. The sensed cardiac signals processed by the sensing circuitry 204 may be received by noise reduction circuitry 203, which may further reduce noise before signals are sent to the detection circuitry 202.

Noise reduction circuitry 203 may also be incorporated after sensing circuitry 202 in cases where high power or computationally intensive noise reduction algorithms are required. The noise reduction circuitry 203, by way of amplifiers used to perform operations with the electrode signals, may also perform the function of the sensing circuitry 204. Combining the functions of sensing circuitry 204 and noise reduction circuitry 203 may be useful to minimize the necessary componentry and lower the power requirements of the system.

In the illustrative configuration shown in FIG. 1E, the detection circuitry 202 is coupled to, or otherwise incorporates, noise reduction circuitry 203. The noise reduction circuitry 203 operates to improve the signal-to-noise ratio (SNR) of sensed cardiac signals by removing noise content of the sensed cardiac signals introduced from various sources. Typical types of transthoracic cardiac signal noise includes electrical noise and noise produced from skeletal muscles, for example.

Detection circuitry 202 typically includes a signal processor that coordinates analysis of the sensed cardiac signals and/or other sensor inputs to detect cardiac arrhythmias, such as, in particular, tachyarrhythmia. Rate based and/or morphological discrimination algorithms may be implemented by the signal processor of the detection circuitry 202 to detect and verify the presence and severity of an arrhythmic episode. Exemplary arrhythmia detection and discrimination circuitry, structures, and techniques, aspects of which may be implemented by an ITCS device of a type that may benefit from disordered breathing detection and/or treatment in accordance with the present invention, are disclosed in commonly owned U.S. Pat. Nos. 5,301,677 and 6,438,410, and in U.S. Pat. Nos. 6,487,443; 6,259,947; 6,141,581; 5,855,593; and 5,545,186, which are hereby incorporated herein by reference.

The detection circuitry 202 communicates cardiac signal information to the control system 205. Memory circuitry 209 of the control system 205 contains parameters for operating in various sensing, defibrillation, and, if applicable, pacing modes, and stores data indicative of cardiac signals received by the detection circuitry 202. The memory circuitry 209 may also be configured to store historical ECG and therapy data, which may be used for various purposes and transmitted to an external receiving device as needed or desired.

In certain configurations, the ITCS device may include diagnostics circuitry 210. The diagnostics circuitry 210 typically receives input signals from the detection circuitry 202 and the sensing circuitry 204. The diagnostics circuitry 210 provides diagnostics data to the control system 205, it being understood that the control system 205 may incorporate all or part of the diagnostics circuitry 210 or its functionality. The control system 205 may store and use information provided by the diagnostics circuitry 210 for a variety of diagnostics purposes. This diagnostic information may be stored, for example, subsequent to a triggering event or at predetermined intervals, and may include system diagnostics, such as power source status, therapy delivery history, and/or patient diagnostics. The diagnostic information may take the form of electrical signals or other sensor data acquired immediately prior to therapy delivery.

According to a configuration that provides cardioversion and defibrillation therapies, the control system 205 processes cardiac signal data received from the detection circuitry 202 and initiates appropriate tachyarrhythmia therapies to terminate cardiac arrhythmic episodes and return the heart to normal sinus rhythm. The control system 205 is coupled to shock therapy circuitry 216. The shock therapy circuitry 216 is coupled to the subcutaneous electrode(s) 214 and the can or indifferent electrode 207 of the ITCS device housing. Upon command, the shock therapy circuitry 216 delivers cardioversion and defibrillation stimulation energy to the heart in accordance with a selected cardioversion or defibrillation therapy. In a less sophisticated configuration, the shock therapy circuitry 216 is controlled to deliver defibrillation therapies, in contrast to a configuration that provides for delivery of both cardioversion and defibrillation therapies. Exemplary ICD high energy delivery circuitry, structures and functionality, aspects of which may be incorporated in an ITCS device of a type that may benefit from aspects of the present invention are disclosed in commonly owned U.S. Pat. Nos. 5,372,606; 5,411,525; 5,468,254; and 5,634,938, which are hereby incorporated herein by reference.

In accordance with another configuration, an ITCS device may incorporate a cardiac pacing capability in addition to cardioversion and/or defibrillation capabilities. As is shown in dotted lines in FIG. 1E, the ITCS device may include pacing therapy circuitry 230, which is coupled to the control system 205 and the subcutaneous and can/indifferent electrodes 214, 207. Upon command, the pacing therapy circuitry delivers pacing pulses to the heart in accordance with a selected pacing therapy. Control signals, developed in accordance with a pacing regimen by pacemaker circuitry within the control system 205, are initiated and transmitted to the pacing therapy circuitry 230 where pacing pulses are generated. A pacing regimen may be modified by the control system 205.

A number of cardiac pacing therapies may be useful in a transthoracic cardiac monitoring and/or stimulation device. Such cardiac pacing therapies may be delivered via the pacing therapy circuitry 230 as shown in FIG. 1E. Alternatively, cardiac pacing therapies may be delivered via the shock therapy circuitry 216, which effectively obviates the need for separate pacemaker circuitry.

The ITCS device shown in FIG. 1E is configured to receive signals from one or more physiologic and/or non-physiologic sensors. Depending on the type of sensor employed, signals generated by the sensors may be communicated to transducer circuitry coupled directly to the detection circuitry 202 or indirectly via the sensing circuitry 204. It is noted that certain sensors may transmit sense data to the control system 205 without processing by the detection circuitry 202.

Non-electrophysiological cardiac sensors may be coupled directly to the detection circuitry 202 or indirectly via the sensing circuitry 204. Non-electrophysiological cardiac sensors sense cardiac activity that is non-electrophysiological in nature. Examples of non-electrophysiological cardiac sensors include blood oxygen sensors, transthoracic impedance sensors, blood volume sensors, acoustic sensors and/or pressure transducers, and accelerometers. Signals from these sensors are developed based on cardiac activity, but are not derived directly from electrophysiological sources (e.g., R-waves or P-waves). A non-electrophysiological cardiac sensor 261, as is illustrated in FIG. 1C, may be connected to one or more of the sensing circuitry 204, detection circuitry 202 (connection not shown for clarity), and the control system 205.

Communications circuitry 218 is coupled to the microprocessor 206 of the control system 205. The communications circuitry 218 allows the ITCS device to communicate with one or more receiving devices or systems situated external to the ITCS device. By way of example, the ITCS device may communicate with a patient-worn, portable or bedside communication system via the communications circuitry 218. In one configuration, one or more physiologic or non-physiologic sensors (subcutaneous, cutaneous, or external of patient) may be equipped with a short-range wireless communication interface, such as an interface conforming to a known communications standard, such as Bluetooth or IEEE 802 standards. Data acquired by such sensors may be communicated to the ITCS device via the communications circuitry 218. It is noted that physiologic or non-physiologic sensors equipped with wireless transmitters or transceivers may communicate with a receiving system external of the patient.

The communications circuitry 218 may allow the ITCS device to communicate with an external programmer. In one configuration, the communications circuitry 218 and the programmer unit (not shown) use a wire loop antenna and a radio frequency telemetric link, as is known in the art, to receive and transmit signals and data between the programmer unit and communications circuitry 218. In this manner, programming commands and data are transferred between the ITCS device and the programmer unit during and after implant. Using a programmer, a physician is able to set or modify various parameters used by the ITCS device. For example, a physician may set or modify parameters affecting sensing, detection, pacing, and defibrillation functions of the ITCS device, including pacing and cardioversion/defibrillation therapy modes.

Typically, the ITCS device is encased and hermetically sealed in a housing suitable for implanting in a human body as is known in the art. Power to the ITCS device is supplied by an electrochemical power source 220 housed within the ITCS device. In one configuration, the power source 220 includes a rechargeable battery. According to this configuration, charging circuitry is coupled to the power source 220 to facilitate repeated non-invasive charging of the power source 220. The communications circuitry 218, or separate receiver circuitry, is configured to receive RF energy transmitted by an external RF energy transmitter. The ITCS device may, in addition to a rechargeable power source, include a non-rechargeable battery. It is understood that a rechargeable power source need not be used, in which case a long-life non-rechargeable battery is employed.

The components, functionality, and structural configurations depicted in FIGS. 1A-1E are intended to provide an understanding of various features and combination of features that may be incorporated in an ITCS device. It is understood that a wide variety of ITCS and other implantable cardiac monitoring and/or stimulation device configurations are contemplated, ranging from relatively sophisticated to relatively simple designs. As such, particular ITCS or cardiac monitoring and/or stimulation device configurations may include particular features as described herein, while other such device configurations may exclude particular features described herein.

In accordance with embodiments of the invention, an ITCS device may be implemented to include a subcutaneous electrode system that provides for one or both of cardiac sensing and arrhythmia therapy delivery. According to one approach, an ITCS device may be implemented as a chronically implantable system that performs monitoring, diagnostic and/or therapeutic functions. The ITCS device may automatically detect and treat cardiac arrhythmias.

In one configuration, an ITCS device includes a pulse generator and one or more electrodes that are implanted subcutaneously in the chest region of the body, such as in the anterior thoracic region of the body. The ITCS device may be used to provide atrial and/or ventricular therapy for bradycardia and tachycardia arrhythmias. Tachyarrhythmia therapy may include cardioversion, defibrillation and anti-tachycardia pacing (ATP), for example, to treat atrial or ventricular tachycardia or fibrillation. Bradycardia therapy may include temporary post-shock pacing for bradycardia or asystole. Methods and systems for implementing post-shock pacing for bradycardia or asystole are described in commonly owned U.S. patent application entitled “Subcutaneous Cardiac Stimulator Employing Post-Shock Transthoracic Asystole Prevention Pacing, Ser. No. 10/377,274, filed on Feb. 28, 2003, which is incorporated herein by reference.

In one configuration, an ITCS device according to one approach may utilize conventional pulse generator and subcutaneous electrode implant techniques. The pulse generator device and electrodes may be chronically implanted subcutaneously. Such an ITCS may be used to automatically detect and treat arrhythmias similarly to conventional implantable systems. In another configuration, the ITCS device may include a unitary structure (e.g., a single housing/unit). The electronic components and electrode conductors/connectors are disposed within or on the unitary ITCS device housing/electrode support assembly.

The ITCS device contains the electronics and may be similar to a conventional implantable defibrillator. High voltage shock therapy may be delivered between two or more electrodes, one of which may be the pulse generator housing (e.g., can), placed subcutaneously in the thoracic region of the body.

Additionally or alternatively, the ITCS device may also provide lower energy electrical stimulation for bradycardia therapy. The ITCS device may provide brady pacing similarly to a conventional pacemaker. The ITCS device may provide temporary post-shock pacing for bradycardia or asystole. Sensing and/or pacing may be accomplished using sense/pace electrodes positioned on an electrode subsystem also incorporating shock electrodes, or by separate electrodes implanted subcutaneously.

The ITCS device may detect a variety of physiological signals that may be used in connection with various diagnostic, therapeutic or monitoring implementations in accordance with the present invention. For example, the ITCS device may include sensors or circuitry for detecting pulse pressure signals, blood oxygen level, heart sounds, cardiac acceleration, and other non-electrophysiological signals related to cardiac activity. In one embodiment, the ITCS device senses intrathoracic impedance, from which various respiratory parameters may be derived, including, for example, respiratory tidal volume and minute ventilation. Sensors and associated circuitry may be incorporated in connection with an ITCS device for detecting one or more body movement or body position related signals. For example, accelerometers and GPS devices may be employed to detect patient activity, patient location, body orientation, or torso position.

The ITCS device may be used within the structure of an APM system. APM systems may allow physicians to remotely and automatically monitor cardiac and respiratory functions, as well as other patient conditions. In one example, implantable cardiac rhythm management systems, such as cardiac pacemakers, defibrillators, and resynchronization devices, may be equipped with various telecommunications and information technologies that enable real-time data collection, diagnosis, and treatment of the patient. Various embodiments described herein may be used in connection with advanced patient management. Methods, structures, and/or techniques described herein, which may be adapted to provide for remote patient/device monitoring, diagnosis, therapy, or other APM related methodologies, may incorporate features of one or more of the following references: U.S. Pat. Nos. 6,221,011; 6,270,457; 6,277,072; 6,280,380; 6,312,378; 6,336,903; 6,358,203; 6,368,284; 6,398,728; and 6,440,066, which are hereby incorporated herein by reference.

An ITCS device according to one approach provides an easy to implant therapeutic, diagnostic or monitoring system. The ITCS system may be implanted without the need for intravenous or intrathoracic access, providing a simpler, less invasive implant procedure and minimizing lead and surgical complications. In addition, this system would have advantages for use in patients for whom transvenous lead systems cause complications. Such complications include, but are not limited to, surgical complications, infection, insufficient vessel patency, complications associated with the presence of artificial valves, and limitations in pediatric patients due to patient growth, among others. An ITCS system according to this approach is distinct from conventional approaches in that it may be configured to include a combination of two or more electrode subsystems that are implanted subcutaneously in the anterior thorax.

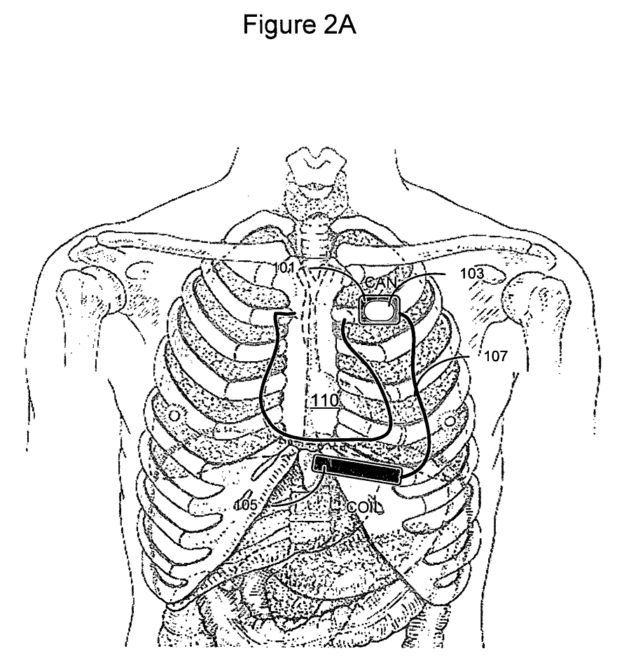

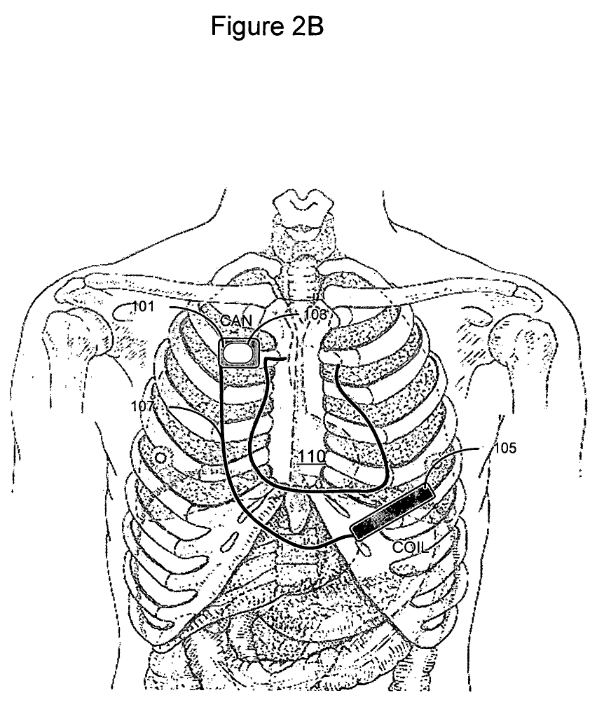

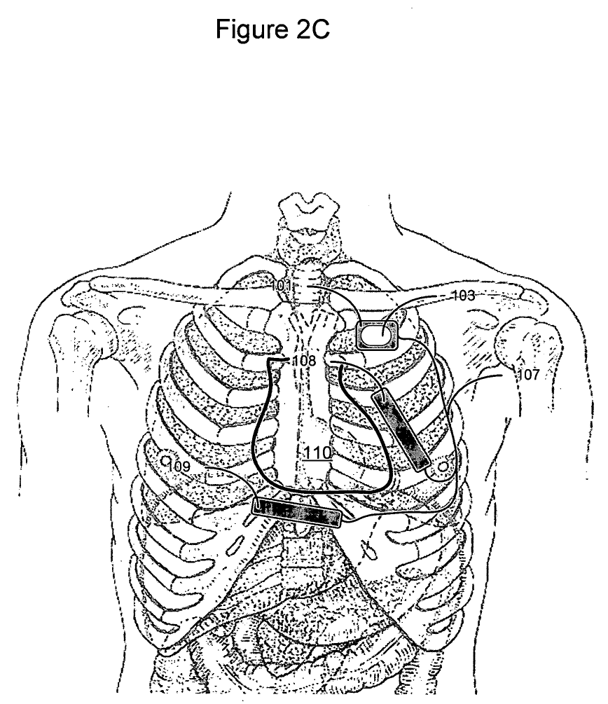

In one configuration, illustrated in FIG. 2A, electrode subsystems of the ITCS system include a first electrode subsystem, including a can electrode 103, and a second electrode subsystem 105 that may include at least one coil electrode, for example. The second electrode subsystem 105 may include a number of electrodes used for sensing and/or electrical stimulation. In various configurations, the second electrode subsystem 105 may include a single electrode or a combination of electrodes. The single electrode or combination of electrodes including the second electrode subsystem 105 may include coil electrodes, tip electrodes, ring electrodes, multi-element coils, spiral coils, spiral coils mounted on non-conductive backing, and screen patch electrodes, for example. A suitable non-conductive backing material is silicone rubber, for example.

The can electrode 103 is located on the housing 101 that encloses the ITCS device electronics. In one embodiment, the can electrode 103 includes the entirety of the external surface of housing 101. In other embodiments, various portions of the housing 101 may be electrically isolated from the can electrode 103 or from tissue. For example, the active area of the can electrode 103 may include all or a portion of either the anterior or posterior surface of the housing 101 to direct current flow in a manner advantageous for cardiac sensing and/or stimulation.

The housing 101 may resemble that of a conventional implantable ICD, is approximately 20-100 cc in volume, with a thickness of 0.4 to 2 cm and with a surface area on each face of approximately 30 to 100 cm². As previously discussed, portions of the housing may be electrically isolated from tissue to optimally direct current flow. For example, portions of the housing 101 may be covered with a non-conductive, or otherwise electrically resistive, material to direct current flow. Suitable non-conductive material coatings include those formed from silicone rubber, polyurethane, or parylene, for example.

FIG. 2A illustrates the housing 101 and can electrode 103 placed subcutaneously, superior to the heart 110 in the left pectoral region, which is a location commonly used for conventional pacemaker and defibrillator implants. The second electrode subsystem 105 may include a coil electrode mounted on the distal end of a lead body 107, where the coil is approximately 3-15 French in diameter and 5-12 cm in length. The coil electrode may have a slight preformed curve along its length. The lead may be introduced through the lumen of a subcutaneous sheath, through a common tunneling implant technique, and the second electrode subsystem 105, e.g., including a coil electrode, may be placed subcutaneously, deep to any subcutaneous fat and adjacent to the underlying muscle layer.

In this configuration, the second electrode subsystem 105 is located approximately parallel with the inferior aspect of the right ventricle of the heart 110, just inferior to the right ventricular free wall, with one end extending just past the apex of the heart 110. For example, the tip of the electrode subsystem 105 may extend less than about 3 cm and may be about 1-2 cm left lateral to the apex of the heart 110. This electrode arrangement may be used to include a majority of ventricular tissue within a volume defined between the housing 101 and the second electrode subsystem 105. In one configuration, a majority of the ventricular tissue is included within a volume associated with an area bounded by lines drawn between the distal and proximal ends of the second electrode subsystem 105 and the medial and lateral edges of the left pectoral can electrode 103.

In one example arrangement, the volume including a majority of ventricular tissue may be associated with a cross sectional area bounded by lines drawn between the ends of the electrode subsystems 103, 105 or between active elements of the electrode subsystems 103, 105. In one implementation, the lines drawn between active elements of the electrode subsystems 103, 105 may include a medial edge and a lateral edge of the can electrode 103, and a proximal end and a distal end of a coil electrode utilized within the second electrode subsystem 105. Arranging the electrode subsystems so that a majority of ventricular tissue is contained within a volume defined between the active elements of the electrode subsystems 103, 105 provides an efficient position for defibrillation by increasing the voltage gradient in the ventricles of the heart 110 for a given applied voltage between electrode subsystems 103, 105.

In a similar configuration, and as shown in FIG. 2B, the housing 101 including the can electrode 103 is placed in the right pectoral region. The second electrode subsystem 105 is located more laterally, to again include a majority of the ventricular tissue in a volume defined between the can electrode 103 and the second electrode subsystem 105.

In a further configuration, and as shown in FIG. 2C, the ITCS device housing 101 containing the electronics (i.e., the can) is not used as an electrode. In this case, an electrode system including two electrode subsystems 108, 109 coupled to the housing 101 may be implanted subcutaneously in the chest region of the body, such as in the anterior thorax. The first and the second electrode subsystems 108, 109 are placed in opposition with respect to the ventricles of the heart 110, with the majority of the ventricular tissue of the heart 110 included within a volume defined between the electrode subsystems 108, 109. As illustrated in FIG. 2C, the first electrode system 108 is located superior to the heart 110 relative to a superior aspect of the heart 110, e.g., parallel to the left ventricular free wall. The second electrode system 109 is located inferior to the heart 110 and positioned in relation to an inferior aspect of the heart 110, e.g., parallel to the right ventricular free wall.

In this configuration, the first and the second electrode subsystems 108, 109 may include any combination of electrodes, including or excluding the can electrode, used for sensing and/or electrical stimulation. In various configurations, the electrode subsystems 108, 109 may each be a single electrode or a combination of electrodes. The electrode or electrodes including the first and second electrode subsystems 108, 109 may include any combination of one or more coil electrodes, tip electrodes, ring electrodes, multi-element coils, spiral coils, spiral coils mounted on non-conductive backing, and screen patch electrodes, for example.

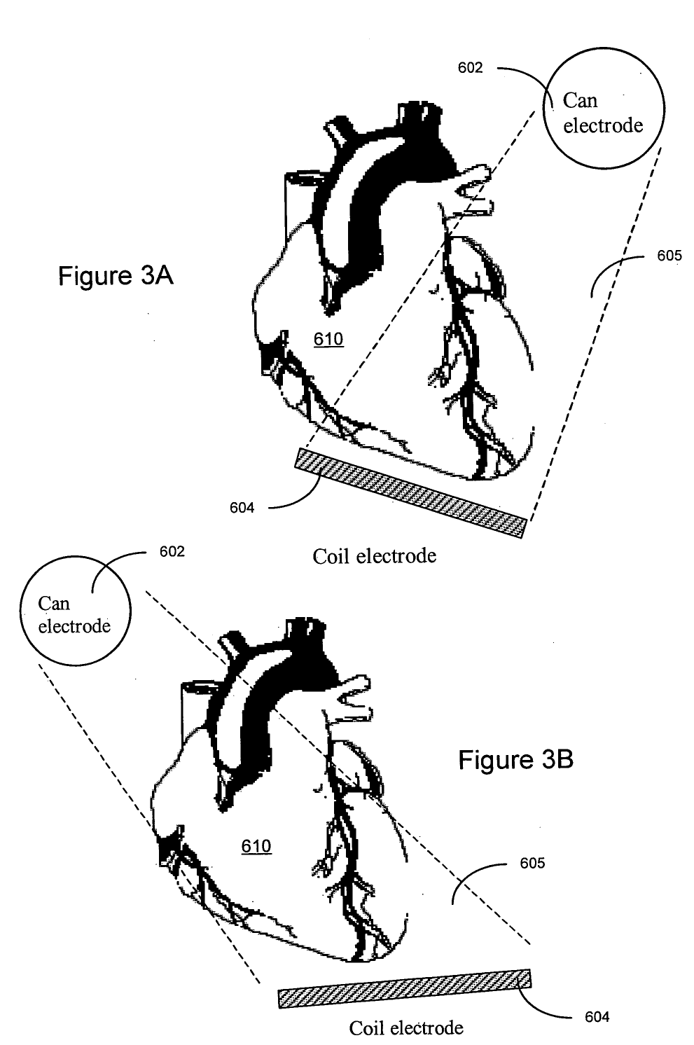

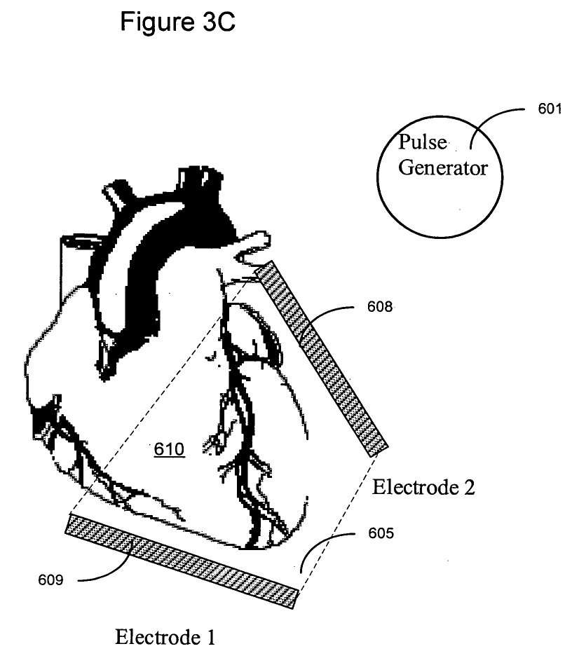

FIGS. 3A-3C provide additional detailed views of subcutaneous electrode subsystem placement considered particularly useful with ITCS devices incorporating disordered breathing detection in accordance with embodiments of the present invention. FIG. 3A illustrates first and second electrode subsystems configured as a can electrode 602 and a coil electrode 604, respectively. FIG. 3A illustrates the can electrode 602 located superior to the heart 610 in the left pectoral region and the coil electrode 604 located inferior to the heart 610, parallel to the right ventricular free wall of the heart 610.

The can electrode 602 and the coil electrode 604 are located so that the majority of ventricular tissue is included within a volume defined between the can electrode 602 and the coil electrode 604. FIG. 3A illustrates a cross sectional area 605 formed by the lines drawn between active elements of the can electrode 602 and the coil electrode 604. Lines drawn between active areas of the electrodes 602, 604, may be defined by a medial edge and a lateral edge of the can electrode 602, and a proximal end and a distal end of a coil electrode utilized as the second electrode subsystem 604. The coil electrode 604 extends a predetermined distance beyond the apex of the heart 610, e.g. less than about 3 cm.

A similar configuration is illustrated in FIG. 3B. In this embodiment, the can electrode 602 is placed superior to the heart 610 in the right pectoral region. The coil electrode 604 is located inferior to the heart. In one arrangement, the coil electrode is located relative to an inferior aspect of the heart 610, for example, the apex of the heart. The can electrode 602 and the coil electrode 604 are positioned so that the majority of ventricular tissue is included within a volume defined between the can electrode 602 and the coil electrode 604.

FIG. 3B illustrates a cross sectional area 605 formed by the lines drawn between active elements of the can electrode 602 and the coil electrode 604. Lines drawn between active areas of the electrodes 602, 604, may be defined by a medial edge and a lateral edge of the can electrode 602, and a proximal end and a distal end of a coil electrode utilized as the second electrode subsystem 604. The coil electrode 604 extends a predetermined distance beyond the apex of the heart 610, e.g. less than about 3 cm.

FIG. 3C illustrates a configuration wherein the pulse generator housing 601 does not include an electrode. In this implementation two electrode subsystems are positioned about the heart so that a majority of ventricular tissue is included within a volume defined between the electrode subsystems. According to this embodiment, the first and second electrodes are configured as first and second coil electrodes 608, 609. The first coil electrode 608 is located superior to the heart 610 and may be located relative to a superior aspect of the heart, e.g., the left ventricular free wall. The second coil electrode 609 is located inferior to the heart 610. The second electrode 609 may be located in relation to an inferior aspect of the heart 610. In one configuration, the second electrode 609 is positioned parallel to the right ventricular free wall with a tip of the electrode 609 extending less than about 3 cm beyond the apex of the heart 610. As illustrated in FIG. 3C, the volume defined between the electrodes may be defined by the cross sectional area 605 bounded by lines drawn between active areas of the electrodes 608, 609.

Various embodiments described herein may be used in connection with the systems and methodologies described in commonly owned U.S. Patent Application Ser. No. 60/504,229 entitled “Methods and Systems for Coordinated Monitoring, Diagnosis, and Therapy,” filed Sep. 18, 2003, which is hereby incorporated herein by reference. Embodiments described herein may be used in connection with detection and/or therapy for disordered breathing. Methods, structures, and/or techniques described herein relating to detection of disordered breathing and therapy to mitigate disordered breathing can incorporate features of one or more of the following commonly owned U.S. patent applications: “Detection of Disordered Breathing,” Ser. No. 10/309,770, filed Dec. 4, 2002; “Prediction of Disordered Breathing,” Ser. No. 10/643,016, filed Aug. 18, 2003; and “Therapy Triggered by Prediction of Disordered Breathing,” Ser. No. 10/643,154, filed Aug. 18, 2003, which are hereby incorporated herein by reference.

Embodiments described herein may be used in connection with sleep detection, sleep quality data collection and evaluation, sleep staging, and sleep informed testing, diagnosis, and/or therapy. Methods, structures, and/or techniques described herein relating to such sleep related processes can incorporate features of one or more of the following commonly owned U.S. patent apps.: “Sleep Detection Using an Adjustable Threshold,” Ser. No. 10/309,771, filed Dec. 4, 2002; and “Sleep State Classification,” Ser. No. 10/643,006, filed Aug. 18, 2003; which are hereby incorporated herein by reference.

Various embodiments described herein may be used in connection with detecting contextual conditions impacting the patient. Methods, structures, and/or techniques described herein relating to contextual condition detection can incorporate features of commonly owned U.S. patent application Ser. No. 10/269,611, filed Oct. 11, 2002, and entitled “Methods and Devices for Detection of Context when Addressing a Medical Condition of a Patient,” which is hereby incorporated herein by reference.

Embodiments described herein may be used in connection with congestive heart failure (CHF) monitoring, diagnosis, and/or therapy. Methods, structures, and/or techniques described herein relating to CHF, such as those involving dual-chamber or bi-ventricular pacing/therapy, cardiac resynchronization therapy, cardiac function optimization, or other CHF related methodologies, can incorporate features of one or more of the following references: commonly owned U.S. patent application Ser. No. 10/270,035, filed Oct. 11, 2002, entitled “Timing Cycles for Synchronized Multisite Cardiac Pacing;” and U.S. Pat. Nos. 6,411,848; 6,285,907; 4,928,688; 6,459,929; 5,334,222; 6,026,320; 6,371,922; 6,597,951; 6,424,865; and 6,542,775, which are hereby incorporated herein by reference.

Various embodiments described herein may be used in connection with preferential pacing/rate regularization therapies. Methods, structures, and/or techniques described herein relating to such therapies, such as those involving single chamber, multi-chamber, multi-site pacing/therapy or other related methodologies, can incorporate features of one or more of the following references: commonly owned U.S. patent application Ser. No. 09/316,515, filed May 21, 1999, entitled “Method and Apparatus for Treating Irregular Ventricular Contractions Such As During Atrial Arrhythmia;” and U.S. Pat. Nos. 6,353,759 and 6,351,669, which are hereby incorporated herein by reference.

Embodiments described herein may be used in connection with approaches to mimic or restore respiratory sinus arrhythmia (RSA). Methods, structures, and/or techniques described herein relating to RSA can incorporate features of U.S. Pat. No. 5,964,788, which is hereby incorporated herein by reference.

Various embodiments described herein may be used in connection with APM systems. Methods, structures, and/or techniques described herein relating to APM, such as those involving remote patient/device monitoring, diagnosis, therapy, or other APM related methodologies, can incorporate features of one or more of the following references: U.S. Pat. Nos. 6,221,011; 6,270,457; 6,277,072; 6,280,380; 6,312,378; 6,336,903; 6,358,203; 6,368,284; 6,398,728; and 6,440,066, which are hereby incorporated herein by reference.

Embodiments described herein may be used in connection with various subcutaneous monitoring, diagnosis, and/or therapy delivery techniques. Methods, structures, and/or techniques described herein relating to such subcutaneous monitoring, diagnosis, and/or therapy delivery processes can incorporate features of one or more of the following references: commonly owned U.S. patent apps.: “Subcutaneous Cardiac Sensing, Stimulation, Lead Delivery, and Electrode Fixation Systems and Methods,” Ser. No. 60/462,272, filed Apr. 11, 2003; “Hybrid Transthoracic/Intrathoracic Cardiac Stimulation Devices and Methods,” Serial No. 10/462,001, filed Jun. 13, 2003; and “Methods and Systems Involving Subcutaneous Electrode Positioning Relative to a Heart,” Ser. No. 10/465,520, filed Jun. 19, 2003; and U.S. Pat. Nos. 5,203,348; 5,230,337; 5,360,442; 5,366,496; 5,397,342; 5,391,200; 5,545,202; 5,603,732; 5,916,243, which are hereby incorporated herein by reference.

Various embodiments described herein may be used in connection with arrhythmia detection, diagnosis, discrimination, and/or therapy. For example, an ITCS device may be used to implement various diagnostic functions, which may involve performing rate-based, pattern and rate-based, and/or morphological tachyarrhythmia discrimination analyses. Subcutaneous, cutaneous, and/or external sensors may be employed to acquire physiologic and non-physiologic information for purposes of enhancing tachyarrhythmia detection and termination. Methods, structures, and/or techniques described herein relating to arrhythmia detection and/or therapy, such as those involving rate- or pattern-based or morphology-based detection, internal and/or external arrhythmia detection and/or therapy, or other arrhythmia related methodologies, can incorporate features of one or more of the following references: commonly owned U.S. patent app. entitled “Cardiac Waveform Template Creation, Maintenance and Use,” Ser. No. 10/448,260, filed May 28, 2003; and U.S. Pat. Nos. 6,449,503; 5,301,677; 6,438,410; 6,487,443; 6,259,947; 6,141,581; 5,855,593; 5,545,186, which are hereby incorporated herein by reference.

Certain embodiments illustrated herein are generally described as capable of implementing various functions traditionally performed by an ICD, and may operate in numerous cardioversion/defibrillation modes as are known in the art. Exemplary ICD circuitry, structures and functionality, aspects of which may be incorporated in an ITCS device of a type that may benefit from disordered breathing detection and/or treatment in accordance with the present invention, are disclosed in commonly owned U.S. Pat. Nos. 5,133,353; 5,179,945; 5,314,459; 5,318,597; 5,620,466; and. 5,662,688, which are hereby incorporated herein by reference.

In particular configurations, systems and methods may perform functions traditionally performed by pacemakers, such as providing various pacing therapies as are known in the art, in addition to cardioversion/defibrillation therapies.

Exemplary pacemaker circuitry, structures and functionality, aspects of which may be incorporated in an ITCS device of a type that may benefit from disordered breathing detection and/or treatment, are disclosed in commonly owned U.S. Pat. Nos. 4,562,841; 5,284,136; 5,376,106; 5,036,849; 5,540,727; 5,836,987; 6,044,298; and 6,055,454, which are hereby incorporated herein by reference. It is understood that ITCS device configurations may provide for non-physiologic pacing support in addition to, or to the exclusion of, bradycardia and/or anti-tachycardia pacing therapies.

An ITCS device in accordance with the present invention may implement diagnostic and/or monitoring functions as well as provide cardiac stimulation therapy. Exemplary cardiac monitoring circuitry, structures and functionality, aspects of which may be incorporated in an ITCS device of a type that may benefit from disordered breathing detection and/or treatment in accordance with the present invention, are disclosed in commonly owned U.S. Pat. Nos. 5,313,953; 5,388,578; and 5,411,031, and in commonly owned U.S. patent application Ser. No. 10/804,471 filed Mar. 19, 2004, entitled “Multiple-Parameter Arrhythmia Discrimination”; and U.S. patent application entitled “Automatic Orientation Determination for ECG Measurements using Multiple Electrodes,” filed Jun. 24, 2004 under Attorney Docket GUID.149PA, which are hereby incorporated herein by reference.

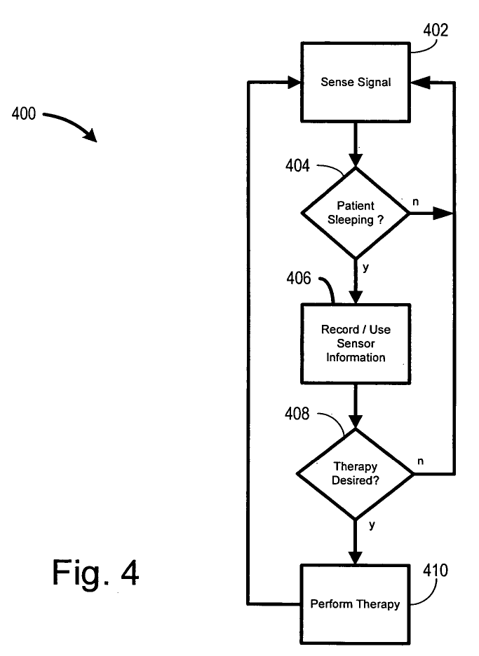

FIG. 4 illustrates a method 400 for implantably sensing and detecting disordered breathing using brain state sensing. A brain state sense signal is sensed at a block 402. Brain state may be sensed, for example, directly using EEG sensors, and/or indirectly using ECG sensors, EEG sensors, EMG sensors, transthoracic impedance sensors, or other sensors suitable for determining patient brain state. If the patient is sleeping, brain state may be detected using the brain state sense signal illustrated by determination block 404.

The brain state detected at determination block 404 provides various types of information recorded at block 406. For example, date, time, sensor data, sense signal amplitudes and/or cycle lengths. This and other information may be useful for updating, developing, and/or determining an arousal index, an apnea/hypopnea index, a composite index and other parameters useful for patient diagnosis and treatment such as the automatic activation of medical processes to treat disordered breathing, for example. The information recorded at block 406 may be useful, for example, to predict, verify, classify, and/or determine the severity of a disordered breathing episode.

If intervention and/or treatment is desired at determination block 408, the intervention and/or treatment may be performed at block 410 before re-starting the method 400. For example, the intervention at block 410 may be the automatic activation of a medical process, modification of a patient's disordered breathing therapy, or other desirable action.

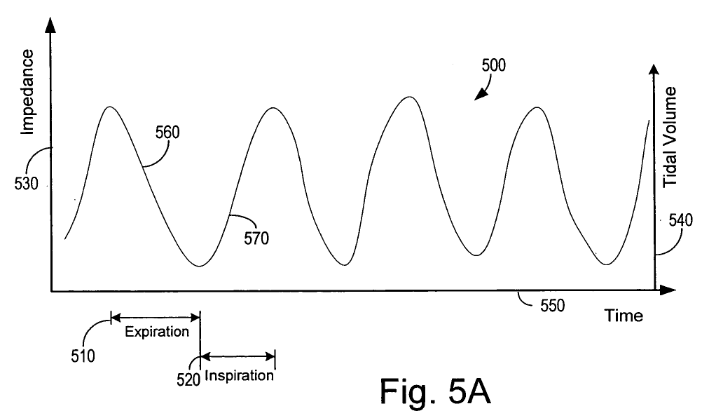

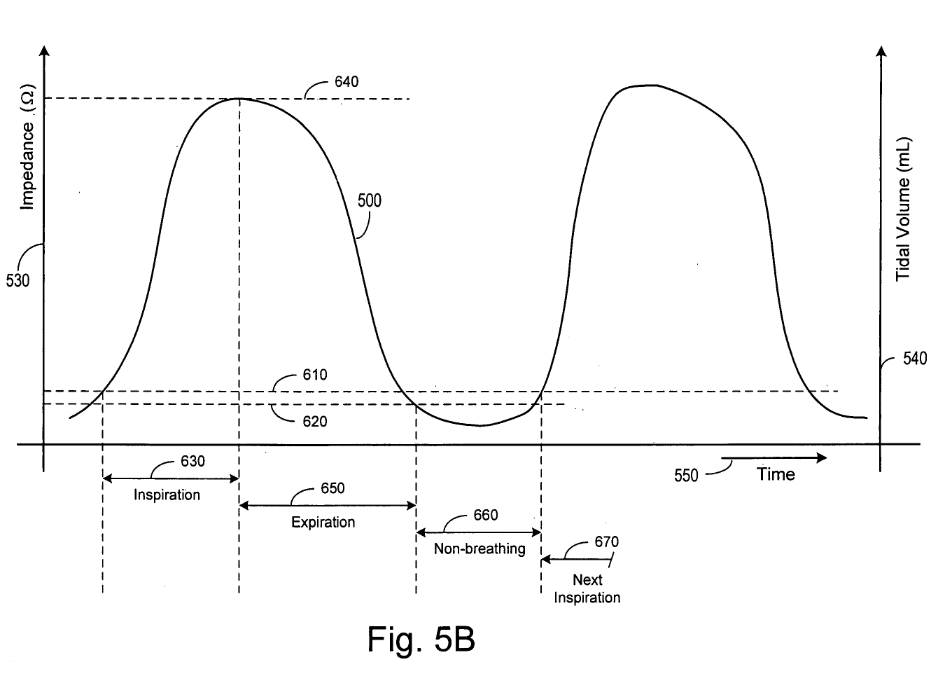

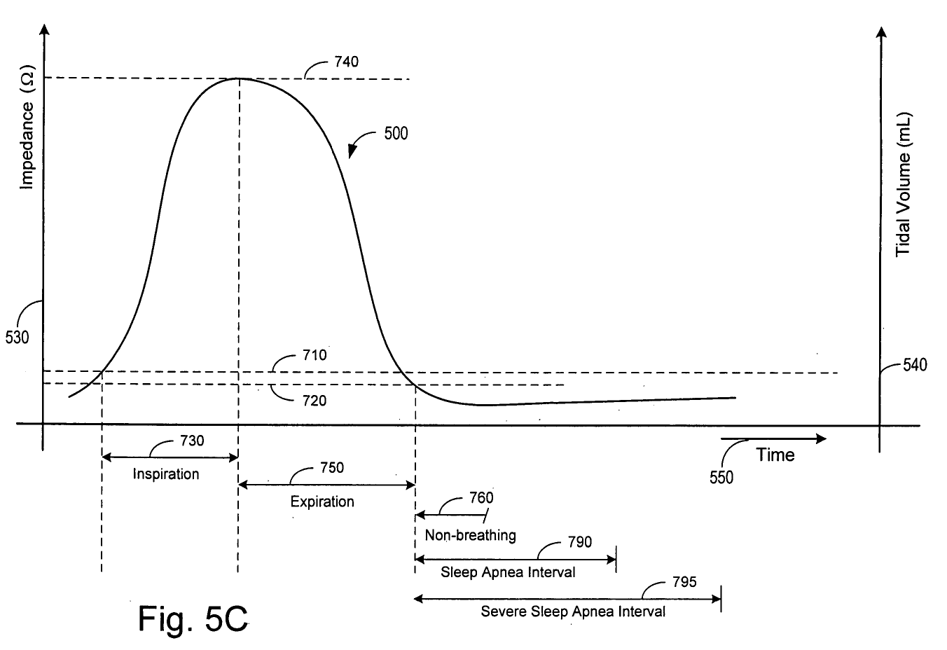

Referring now to FIG. 5A, an impedance signal 500 is illustrated. Transthoracic impedance may be useful for detecting sleep-state and other indirect measurements of brain activity, such as seizures, as well as breathing disorders. The impedance signal 500 may be developed, for example, from an impedance sense electrode in combination with a ITCS device. The impedance signal 500 is proportional to the transthoracic impedance, illustrated as an Impedance 530 on the abscissa of the left side of the graph in FIG. 5A.

The impedance 530 increases during any respiratory inspiration 520 and decreases during any respiratory expiration 510. The impedance signal 500 is also proportional to the amount of air inhaled, denoted by a tidal volume 540, illustrated on the abscissa of the right side of the graph in FIG. 5A. The variations in impedance during respiration, identifiable as the peak-to-peak variation of the impedance signal 500, may be used to determine the respiration tidal volume 540. Tidal volume 540 corresponds to the volume of air moved in a breath, one cycle of expiration 510 and inspiration 520. A minute ventilation may also be determined, corresponding to the amount of air moved per a minute of time 550 illustrated on the ordinate of the graph in FIG. 5A.

The onset of breathing disorders may be determined using the impedance signal 530, and detected breathing disorder information may be used to activate therapy in accordance with the present invention. During non-REM sleep, a normal respiration pattern includes regular, rhythmic inspiration—expiration cycles without substantial interruptions. When the tidal volume of the patient's respiration, as indicated by the transthoracic impedance signal, falls below a hypopnea threshold, then a hypopnea event is declared. For example, a hypopnea event may be declared if the patient's tidal volume falls below about 50% of a recent average tidal volume or other baseline tidal volume value. If the patient's tidal volume falls further to an apnea threshold, e.g., about 10% of the recent average tidal volume or other baseline value, an apnea event is declared.

An adequate quality and quantity of sleep is required to maintain physiological homeostasis. Prolonged sleep deprivation or periods of highly fragmented sleep ultimately has serious health consequences. Chronic lack of sleep may be associated with various cardiac or respiratory disorders affecting a patient's health and quality of life. Methods and systems for collecting and assessing sleep quality data are described in commonly owned U.S. patent application Ser. No. 10/642,998, entitled “Sleep Quality Data Collection and Evaluation,” filed on Aug. 18, 2003, and hereby incorporated herein by reference. Evaluation of the patient's sleep patterns and sleep quality may be an important aspect of providing coordinated therapy to the patient, including respiratory and cardiac therapy.

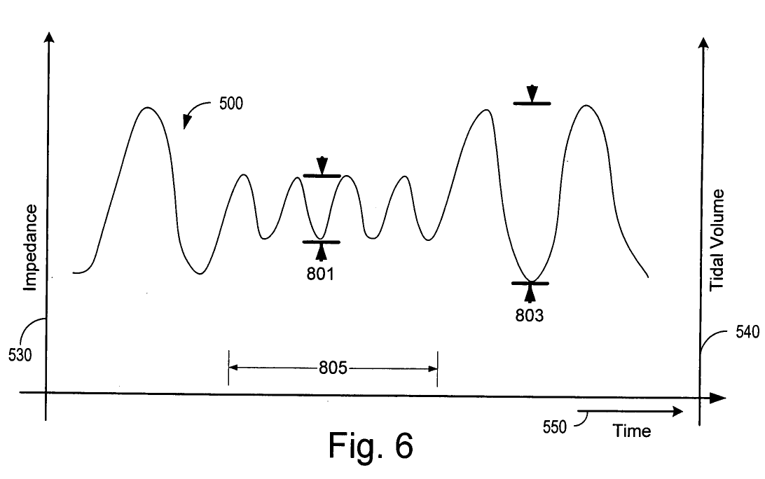

FIGS. 5A, 5B, and 6 are graphs of transthoracic impedance and tidal volume, similar to FIG. 5A previously described. As stated earlier, using transthoracic impedance is one indirect method of determining brain state, such as by detecting sleep state, arousal, and disordered breathing, for example. As in FIG. 5A, FIGS. 5B, 5C and 6, illustrate the impedance signal 500 proportional to the transthoracic impedance, again illustrated as Impedance 530 on the abscissa of the left side of the graphs in FIGS. 5A, 5B, and 6. The impedance 530 increases during any respiratory inspiration 520 and decreases during any respiratory expiration 510. As before, the impedance signal 500 is also proportional to the amount of air inhaled, denoted the tidal volume 540, illustrated on the abscissa of the right side of the graph in FIGS. 5A, 5B, and 6. The magnitude of variations in impedance and tidal volume during respiration are identifiable as the peak-to-peak variation of the impedance signal 500.

FIG. 5B illustrates respiration intervals used for disordered breathing detection useful in accordance with embodiments of the invention. Respiration intervals are used to detect apnea and hypopnea, as well as provide other sleep-state information for activating therapy in accordance with embodiments of the present invention. Detection of disordered breathing may involve defining and examining a number of respiratory cycle intervals. A respiration cycle is divided into an inspiration period corresponding to the patient inhaling, an expiration period, corresponding to the patient exhaling, and a non-breathing period occurring between inhaling and exhaling. Respiration intervals are established using an inspiration threshold 610 and an expiration threshold 620. The inspiration threshold 610 marks the beginning of an inspiration period 630 and is determined by the transthoracic impedance signal 500 rising above the inspiration threshold 610. The inspiration period 630 ends when the transthoracic impedance signal 500 is a maximum 640. The maximum transthoracic impedance signal 640 corresponds to both the end of the inspiration interval 630 and the beginning of an expiration interval 650. The expiration interval 650 continues until the transthoracic impedance 500 falls below an expiration threshold 620. A non-breathing interval 660 starts from the end of the expiration period 650 and continues until the beginning of a next inspiration period 670.

Detection of sleep apnea and severe sleep apnea is illustrated in FIG. 5C. The patient's respiration signals are monitored and the respiration cycles are defined according to an inspiration 730, an expiration 750, and a non-breathing 760 interval as described in connection with FIG. 5B. A condition of sleep apnea is detected when a non-breathing period 760 exceeds a first predetermined interval 790, denoted the sleep apnea interval. A condition of severe sleep apnea is detected when the non-breathing period 760 exceeds a second predetermined interval 795, denoted the severe sleep apnea interval. For example, sleep apnea may be detected when the non-breathing interval exceeds about 10 seconds, and severe sleep apnea may be detected when the non-breathing interval exceeds about 20 seconds.

Hypopnea is a condition of disordered breathing characterized by abnormally shallow breathing. Hypopnea reduces oxygen to the brain, and is linked with altered brain activity and brain states. The altered brain activity and brain states indicative of hypopnea may be used by an ITCS device to activate therapy in accordance with embodiments of the present invention. FIG. 6 is a graph of tidal volume derived from transthoracic impedance measurements. The graph of FIG. 6 illustrating the tidal volume of a hypopnea episode may be compared to the tidal volume of a normal breathing cycle illustrated previously in FIG. 5A, which illustrated normal respiration tidal volume and rate. As shown in FIG. 6, hypopnea involves a period of abnormally shallow respiration, possible at an increased respiration rate.

Hypopnea is detected by comparing a patient's respiratory tidal volume 803 to a hypopnea tidal volume 801. The tidal volume for each respiration cycle may be derived from transthoracic impedance measurements acquired in the manner described previously. The hypopnea tidal volume threshold may be established by, for example, using clinical results providing a representative tidal volume and duration of hypopnea events. In one configuration, hypopnea is detected when an average of the patient's respiratory tidal volume taken over a selected time interval falls below the hypopnea tidal volume threshold. Furthermore, various combinations of hypopnea cycles, breath intervals, and non-breathing intervals may be used to detect hypopnea, where the non-breathing intervals are determined as described above.

In FIG. 6, a hypopnea episode 805 is identified when the average tidal volume is significantly below the normal tidal volume. In the example illustrated in FIG. 6, the normal tidal volume during the breathing process is identified as the peak-to peak value identified as the respiratory tidal volume 803. The hypopnea tidal volume during the hypopnea episode 805 is identified as hypopnea tidal volume 801. For example, the hypopnea tidal volume 801 may be about 50% of the respiratory tidal volume 803. The value 50% is used by way of example only, and determination of thresholds for hypopnea events may be determined as any value appropriate for a given patient.

In the example above, if the tidal volume falls below 50% of the respiratory tidal volume 803, the breathing episode may be identified as a hypopnea event, originating the measurement of the hypopnea episode 805.

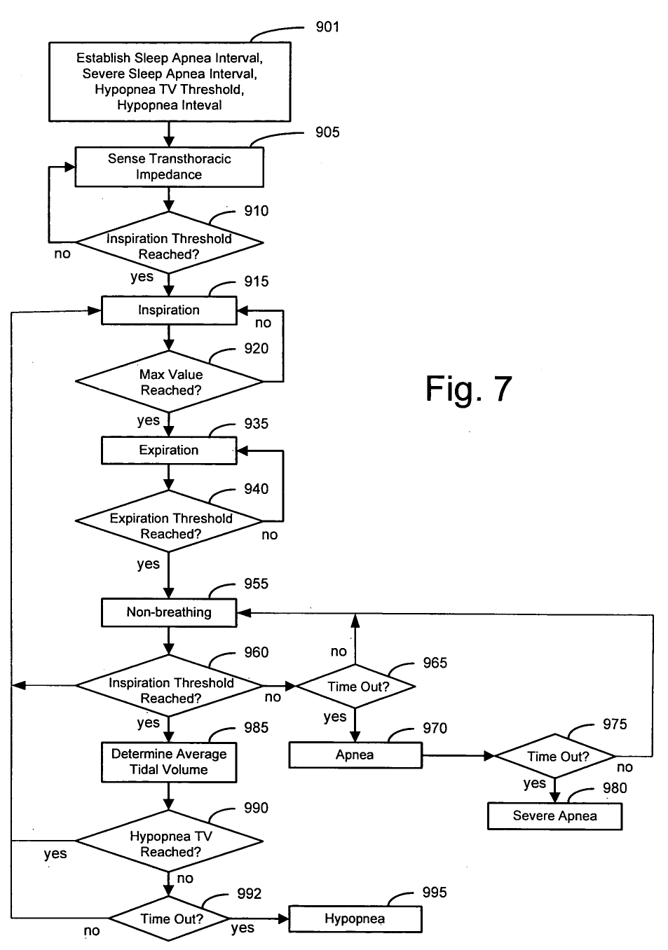

FIG. 7 is a flow chart illustrating a method of apnea and/or hypopnea detection useful for activating therapy based on brain activity in accordance with embodiments of the invention. Various parameters are established 901 before analyzing the patient's respiration for disordered breathing episodes, including, for example, inspiration and expiration thresholds, sleep apnea interval, severe sleep apnea interval, and hypopnea tidal volume (TV) threshold.

The patient's transthoracic impedance is measured 905 as described in more detail above. If the transthoracic impedance exceeds 910 the inspiration threshold, the beginning of an inspiration interval is detected 915. If the transthoracic impedance remains below 910 the inspiration threshold, then the impedance signal is checked 905 periodically until inspiration 915 occurs.

During the inspiration interval, the patient's transthoracic impedance is monitored until a maximum value of the transthoracic impedance is detected 920. Detection of the maximum value signals an end of the inspiration period and a beginning of an expiration period 935.

The expiration interval is characterized by decreasing transthoracic impedance. When, at determination 940, the transthoracic impedance falls below the expiration threshold, a non-breathing interval is detected 955.

If the transthoracic impedance determination 960 does not exceed the inspiration threshold within a first predetermined interval, denoted the sleep apnea interval 965, then a condition of sleep apnea is detected 970. Severe sleep apnea 980 is detected if the non-breathing period extends beyond a second predetermined interval, denoted the severe sleep apnea interval 975.

When the transthoracic impedance determination 960 exceeds the inspiration threshold, the tidal volume from the peak-to-peak transthoracic impedance is calculated, along with a moving average of past tidal volumes 985. The peak-to-peak transthoracic impedance provides a value proportional to the tidal volume of the respiration cycle. This value is compared at determination 990 to a hypopnea tidal volume threshold. If, at determination 990, the peak-to-peak transthoracic impedance is consistent with the hypopnea tidal volume threshold for a predetermined time 992, then a hypopnea cycle 995 is detected.

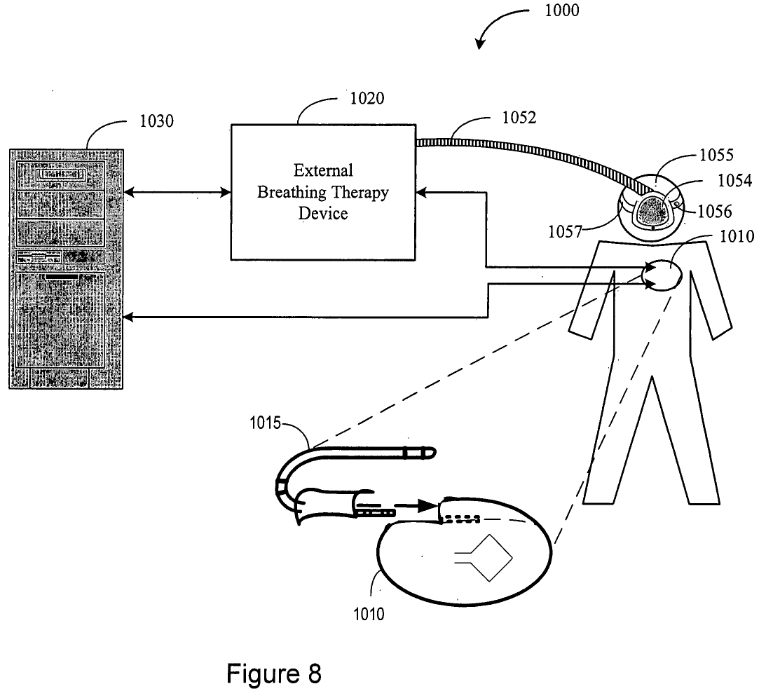

According to one embodiment of the invention, illustrated in FIG. 8, a medical system 1000 may include an ITCS 1010 that cooperates with a patient-external respiration therapy device 1020 to provide coordinated patient monitoring, diagnosis and/or therapy. In the example illustrated in FIG. 8, a mechanical respiration therapy device, designated CPAP device 1020, includes a positive airway pressure device that cooperates with the ITCS 1010. Positive airway pressure devices may be used to provide a variety of respiration therapies, including, for example, continuous positive airway pressure (CPAP), bi-level positive airway pressure (bi-level PAP), proportional positive airway pressure (PPAP), auto-titrating positive airway pressure, ventilation, gas or oxygen therapies. These therapies may be activated, by the ITCS device 1010, based on disordered breathing detection in accordance with embodiments of the present invention.

The CPAP device 1020 develops a positive air pressure that is delivered to the patient's airway through a tube system 1052 and a mask 1054 connected to the CPAP device 1020. The mask 1054 may include EEG sensors, such as an EEG sensor 1056 attached to a strap 1057 that is placed around a head 1055 of the patient. Positive airway pressure devices are often used to treat disordered breathing. In one configuration, for example, the positive airway pressure provided by the CPAP device 1020 acts as a pneumatic splint keeping the patient's airway open and reducing the severity and/or number of occurrences of disordered breathing due to airway obstruction.

The CPAP device 1020 may directly control the delivery of respiration therapy to the patient, and may contribute to the control of the ITCS 1010. In addition, the CPAP device 1020 may provide a number of monitoring and/or diagnostic functions in relation to the respiratory system and/or other physiological systems.

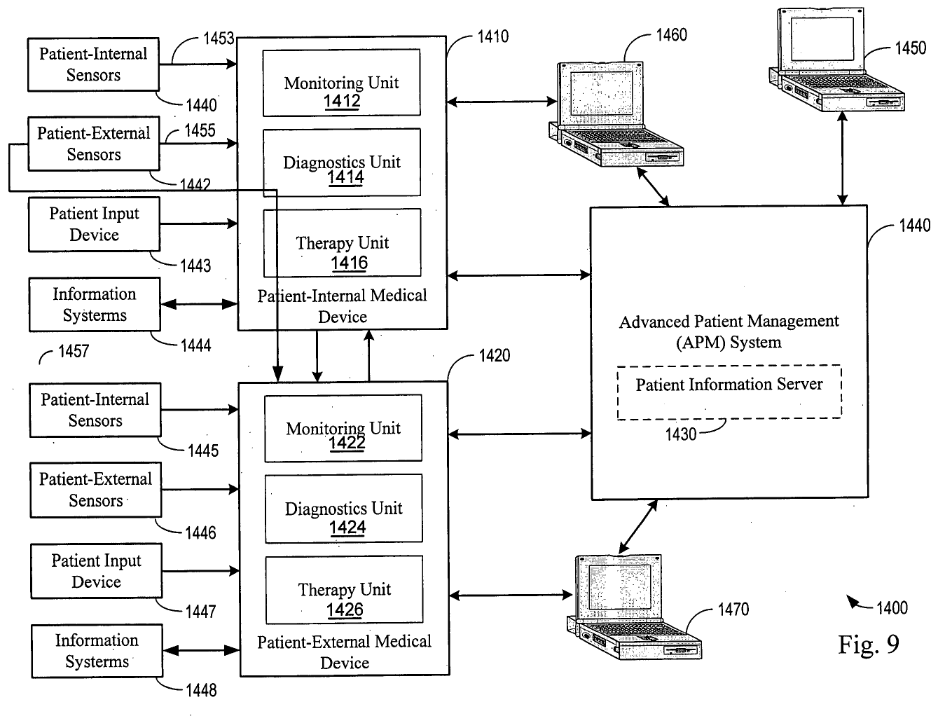

The ITCS 1010 and CPAP 1020 devices may communicate directly through a wireless communications link 1017, for example. Alternatively, or additionally, the ITCS 1010 and CPAP 1020 devices may communicate with and/or through an APM such as an APM system 1030, as will be described further below with reference to FIG. 9. The ITCS 1010 may be electrically coupled to a heart 1040 of the patient using a subcutaneous electrode system 1015, for example.

The ITCS 1010 may provide a first set of monitoring, diagnostic, and/or therapeutic functions to a patient 1055. The ITCS 1010 may be electrically coupled to a patient's heart 1040 through one or more cardiac electrodes 1015. The cardiac electrodes 1015 may sense cardiac signals produced by the heart 1040 and/or provide therapy. The ITCS 1010 may directly control delivery of one or more cardiac therapies, such as cardiac pacing, defibrillation, cardioversion, cardiac resynchronization, and/or other cardiac therapies, for example. In addition, the ITCS 1010 may facilitate the control of a mechanical respiration device 1020. Further, the ITCS 1010 may perform various monitoring and/or diagnostic functions in relation to the cardiovascular system and/or other physiological systems.

Although FIG. 8 illustrates a ITCS device 1010 used with a CPAP device 1020 to provide coordinated patient monitoring, diagnosis and/or therapy, any number of patient-internal and patient-external medical devices may be included in a medical system in accordance with the invention. For example, a drug delivery device, such as a drug pump or controllable nebulizer, may be included in the system 1000. The drug delivery device may cooperate with either or both of the ITCS device 1010 and the CPAP device 1020 and may contribute to the patient monitoring, diagnosis, and/or therapeutic functions of the medical system 1000.

FIG. 9 is a block diagram of a medical system 1400 that may be used to implement coordinated patient measuring and/or monitoring, diagnosis, and/or therapy, including detecting disordered breathing using an ITCS device in accordance with embodiments of the invention. The medical system 1400 may include, for example, one or more patient-internal medical devices 1410 and one or more patient-external medical devices 1420. Each of the patient-internal 1410 and patient-external 1420 medical devices may include one or more of a patient monitoring unit 1412, 1422, a diagnostics unit 1414, 1424, and/or a therapy unit 1416, 1426.

The patient-internal medical device 1410 is typically a fully or partially implantable device that performs measuring, monitoring, diagnosis, and/or therapy functions. The patient-external medical device 1420 performs monitoring, diagnosis and/or therapy functions external to the patient (i.e., not invasively implanted within the patient's body). The patient-external medical device 1420 may be positioned on the patient, near the patient, or in any location external to the patient. It is understood that a portion of a patient-external medical device 1420 may be positioned within an orifice of the body, such as the nasal cavity or mouth, yet may be considered external to the patient (e.g., mouth pieces/appliances, tubes/appliances for nostrils, or temperature sensors positioned in the ear canal).

The patient-internal and patient-external medical devices 1410, 1420 may be coupled to one or more sensors 1441, 1442, 1445, 1446, patient input devices 1443, 1447 and/or other information acquisition devices 1444, 1448. The sensors 1441, 1442, 1445, 1446, patient input devices 1443, 1447, and/or other information acquisition devices 1444, 1448 may be employed to detect conditions relevant to the monitoring, diagnostic, and/or therapeutic functions of the patient-internal and patient-external medical devices 1410, 1420.

The medical devices 1410, 1420 may each be coupled to one or more patient-internal sensors 1441, 1445 that are fully or partially implantable within the patient. The medical devices 1410, 1420 may also be coupled to patient-external sensors positioned on, near, or in a remote location with respect to the patient. For example, the patient-external sensors 1442 may include EEG sensors useful for detecting brain activity, and airflow sensors or expired gas sensors for detecting breathing irregularities. The patient-internal and patient-external sensors may also be used to sense conditions, such as physiological or environmental conditions, that affect the patient.

The patient-internal sensors 1441 may be coupled to the patient-internal medical device 1410 through one or more internal leads 1453. In one example, as was described above with reference to FIG. 9, an internal endocardial lead system is used to couple cardiac electrodes to an implantable pacemaker or other cardiac rhythm management device. Still referring to FIG. 9, one or more patient-internal sensors 1441 may be equipped with transceiver circuitry to support wireless communications between the one or more patient-internal sensors 1441 and the patient-internal medical device 1410 and/or the patient-external medical device 1420.

The patient-external sensors 1442 may be coupled to the patient-internal medical device 1410 and/or the patient-external medical device 1420 through one or more internal leads 1455 or through wireless connections. Patient-external sensors 1442 may communicate with the patient-internal medical device 1410 wirelessly. Patient-external sensors 1446 may be coupled to the patient-external medical device 1420 through one or more internal leads 1457 or through a wireless link.

The medical devices 1410, 1420 may be coupled to one or more patient input devices 1443, 1447. The patient input devices are used to allow the patient to manually transfer information to the medical devices 1410, 1420. The patient input devices 1443, 1447 may be particularly useful for inputting information concerning patient perceptions, such as how well the patient feels, and information such as patient smoking, drug use, or other activities that are not automatically sensed or detected by the medical devices 1410, 1420.

The medical devices 1410, 1420 may be connected to one or more information acquisition devices 1444, 1448, for example, a database that stores information useful in connection with the monitoring, diagnostic, or therapy functions of the medical devices 1410, 1420. For example, one or more of the medical devices 1410, 1420 may be coupled through a network to a patient information server 1430 that provides information about environmental conditions affecting the patient, e.g., the pollution index for the patient's location.

In one embodiment, the patient-internal medical device 1410 and the patient-external medical device 1420 may communicate through a wireless link between the medical devices 1410, 1420. For example, the patient-internal and patient-external devices 1410, 1420 may be coupled through a short-range radio link, such as Bluetooth, IEEE 802.11, and/or a proprietary wireless protocol. The communications link may facilitate unidirectional or bi-directional communication between the patient-internal 1410 and patient-external 1420 medical devices. Data and/or control signals may be transmitted between the patient-internal 1410 and patient-external 1420 medical devices to coordinate the functions of the medical devices 1410, 1420.

In another embodiment, the patient-internal and patient-external medical devices 1410, 1420 may be used within the structure of an advanced patient management system 1440. Advanced patient management systems 1440 involve a system of medical devices that are accessible through various communications technologies. For example, patient data may be downloaded from one or more of the medical devices periodically or on command, and stored at the patient information server 1430. The physician and/or the patient may communicate with the medical devices and the patient information server 1430, for example, to acquire patient data or to initiate, terminate or modify therapy.

The data stored on the patient information server 1430 may be accessible by the patient and the patient's physician through one or more terminals 1450, e.g., remote computers located in the patient's home or the physician's office. The patient information server 1430 may be used to communicate to one or more of the patient-internal and patient-external medical devices 1410, 1420 to provide remote control of the monitoring, diagnosis, and/or therapy functions of the medical devices 1410, 1420.

In one embodiment, the patient's physician may access patient data (e.g., disordered breathing data) transmitted from the medical devices 1410, 1420 to the patient information server 1430. After evaluation of the patient data, the patient's physician may communicate with one or more of the patient-internal or patient-external devices 1410, 1420 through the APM system 1440 to initiate, terminate, or modify the monitoring, diagnostic, and/or therapy functions of the patient-internal and/or patient-external medical systems 1410, 1420 (e.g., XPAP therapy or cardiac electrical therapy to treat disordered breathing).

In another embodiment, the patient-internal and patient-external medical devices 1410, 1420 may not communicate directly, but may communicate indirectly through the APM system 1440. In this embodiment, the APM system 1440 may operate as an intermediary between two or more of the medical devices 1410, 1420. For example, data and/or control information may be transferred from one of the medical devices 1410, 1420 to the APM system 1440. The APM system 1440 may transfer the data and/or control information to another of the medical devices 1410, 1420.

In one embodiment, the APM system 1440 may communicate directly with the patient-internal and/or patient-external medical devices 1410, 1420. In another embodiment, the APM system 1440 may communicate with the patient-internal and/or patient-external medical devices 1410, 1420 through medical device programmers 1460, 1470 respectively associated with each medical device 1410, 1420.

A number of the examples presented herein involve block diagrams illustrating functional blocks used for coordinated monitoring, diagnosis and/or therapy functions in accordance with embodiments of the invention. It will be understood by those skilled in the art that there exist many possible configurations in which these functional blocks may be arranged and implemented. The examples depicted herein provide examples of possible functional arrangements used to implement the approaches of the invention.

Each feature disclosed in this specification (including any accompanying claims, abstract, and drawings), may be replaced by alternative features having the same, equivalent or similar purpose, unless expressly stated otherwise. Thus, unless expressly stated otherwise, each feature disclosed is one example only of a generic series of equivalent or similar features.

Various modifications and additions can be made to the preferred embodiments discussed hereinabove without departing from the scope of the present invention. Accordingly, the scope of the present invention should not be limited by the particular embodiments described above, but should be defined only by the claims set forth below and equivalents thereof.

1. A system, comprising:
a lead system configured for subcutaneous, non-intrathoracic placement relative to a heart of a patient; and
an implantable device, the implantable device comprising cardiac activity detection circuitry and disordered breathing detection circuitry, the cardiac activity detection circuitry coupled to the lead system and configured to detect cardiac rhythms, and the disordered breathing detection circuitry coupled to the lead system and configured to detect disordered breathing.

2. The system of claim 1, wherein the implantable device further comprises cardiac therapy circuitry coupled to the lead system and configured to deliver a cardiac therapy to treat detected disordered breathing.

3. The system of claim 1, wherein the implantable device further comprises disordered breathing therapy circuitry coupled to the lead system.

4. The system of claim 1, wherein the disordered breathing therapy circuitry comprises circuitry to coordinate delivery of a diaphragmatic pacing therapy.

5. The system of claim 1, wherein the lead system further comprises a hypoglossal nerve lead, and the disordered breathing therapy circuitry is coupled to the hypoglossal nerve lead and comprises circuitry to coordinate delivery of a hypoglossal nerve stimulation therapy.

6. The system of claim 1, further comprising a patient-external respiratory device configured to deliver a disordered breathing therapy to the patient.

7. The system of claim 6, wherein the respiratory device comprises a positive airway pressure device.

8. The system of claim 6, wherein each of the implantable device and the respiratory device comprises communication circuitry configured to facilitate communication between the implantable device and the respiratory device.

9. The system of claim 6, further comprising a patient-external processing system communicatively coupled to the implantable device and the respiratory device, the processing system configured to cooperate with one or both of the implantable device and the respiratory device to coordinate one or more of patient monitoring, diagnosis, and therapy.

10. The system of claim 1, further comprising one or both of a patient-internal drug delivery device or a patient-external drug delivery device.

11. The system of claim 1, further comprising a gas therapy device.

12. The system of claim 1, wherein the disordered breathing detection circuitry comprises an accelerometer configured to detect the patient's respiration.

13. The system of claim 1, wherein the disordered breathing detection circuitry comprises a transthoracic impedance sensor.

14. A method, comprising:
detecting cardiac activity of a patient from subcutaneous, non-intrathoracic locations;
sensing, from one or more subcutaneous, non-intrathoracic locations, one or more physiologic parameters associated with respiration of the patient; and
determining presence of disordered breathing using the sensed one or more physiologic parameters.

15. The method of claim 14, further comprising delivering a cardiac therapy in response to determining presence of disordered breathing.

16. The method of claim 14, further comprising delivering a disordered breathing therapy in response to determining presence of disordered breathing.

17. The method of claim 14, further comprising delivering a diaphragmatic pacing therapy in response to determining presence of disordered breathing.

18. The method of claim 14, further comprising delivering a hypoglossal nerve stimulation therapy in response to determining presence of disordered breathing.

19. A system, comprising:
means for detecting cardiac activity of a patient from subcutaneous, non-intrathoracic locations;
means for sensing, from one or more subcutaneous, non-intrathoracic locations, one or more physiologic parameters associated with respiration of the patient; and
means for determining presence of disordered breathing using the sensed one or more physiologic parameters.

20. The system of claim 20, further comprising means for delivering a disordered breathing therapy in response to determining presence of disordered breathing.
